SEQUENCE LISTING (15 sequences)

SEQ ID NO: 1 - DNA, 1377 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatggcgccacccctgtcgatgaccgacggcgtagctcaagtgaatacccaggacagcaatgcctgggtcgaaatcaataaagccgcgttcgagcacaacatacggactctgcaaaccgccctcgccggcaagtcgcagatctgcgccgtactcaaggcggatgcctatggccacggtatcggcttgttgatgccctcggtgatcgccatgggtgttccctgtgtcggtgtcgccagcaacgaagaagcccgcgtcgtgcgcgagagcggtttcaagggtcaactgatacgcgtgcgcaccgctgccctgagcgaactggaagctgcactgccgtacaacatggaagagctggtgggcaacctggacttcgcggtcaaggccagcctgattgccgaggatcacggtcgcccgctggtggtgcacctgggtctgaattccagcggcatgagccgtaacggagtggacatgaccaccgctcagggccgtcgtgatgcggtagctatcaccaaggtgccaaacctggaagtgcgggcgatcatgacccacttcgcggtcgaagatgctgccgacgtgcgtgccgggctcaaggccttcaatcagcaagcccaatggctgatgaacgtggcccagcttgatcgcagcaagatcaccctgcacgcggccaactcgttcgccacactggaggtgcccgaatcgcatctggacatggtccgccccggcggcgcgctgttcggcgacaccgtaccgtcccacaccgagtacaagcgggtcatgcagttcaagtcccacgtggcgtcggtcaacagctaccccaagggcaacaccgtcggttatggccgcacgtacaccctggggcgcgactcgcggctggccaacatcaccgtcggctactctgacggctaccgccgcgcgtttaccaataaagggattgtgctgatcaacggccatcgcgtgccagtggtgggcaaagtctcgatgaacaccctgatggtggacgtcactgacgcgccggatgtgaaaagcggcgatgaagtggtgctgttcgggcaccagggcaaggccgagattacccaggctgagatcgaagacatcaacggtgcactgcttgcggatctgtataccgtgtggggcaattccaaccctaaaatcctgaaagatcagtaa

SEQ ID NO: 2 - DNA, 213 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 3 - DNA, 43 bp, Artificial Sequence
  Description of Artificial Sequence Synthetic oligonucleotide
ctctagaaataattttgtttaactttaagaaggagatatacat

SEQ ID NO: 4 - DNA, 1164 bp, Artificial Sequence
  Description of Artificial Sequence Synthetic polynucleotide
atggcgccacccctgtcgatgaccgacggcgtagctcaagtgaatacccaggacagcaatgcctgggtcgaaatcaataaagccgcgttcgagcacaacatacggactctgcaaaccgccctcgccggcaagtcgcagatctgcgccgtactcaaggcggatgcctatggccacggtatcggcttgttgatgccctcggtgatcgccatgggtgttccctgtgtcggtgtcgccagcaacgaagaagcccgcgtcgtgcgcgagagcggtttcaagggtcaactgatacgcgtgcgcaccgctgccctgagcgaactggaagctgcactgccgtacaacatggaagagctggtgggcaacctggacttcgcggtcaaggccagcctgattgccgaggatcacggtcgcccgctggtggtgcacctgggtctgaattccagcggcatgagccgtaacggagtggacatgaccaccgctcagggccgtcgtgatgcggtagctatcaccaaggtgccaaacctggaagtgcgggcgatcatgacccacttcgcggtcgaagatgctgccgacgtgcgtgccgggctcaaggccttcaatcagcaagcccaatggctgatgaacgtggcccagcttgatcgcagcaagatcaccctgcacgcggccaactcgttcgccacactggaggtgcccgaatcgcatctggacatggtccgccccggcggcgcgctgttcggcgacaccgtaccgtcccacaccgagtacaagcgggtcatgcagttcaagtcccacgtggcgtcggtcaacagctaccccaagggcaacaccgtcggttatggccgcacgtacaccctggggcgcgactcgcggctggccaacatcaccgtcggctactctgacggctaccgccgcgcgtttaccaataaagggattgtgctgatcaacggccatcgcgtgccagtggtgggcaaagtctcgatgaacaccctgatggtggacgtcactgacgcgccggatgtgaaaagcggcgatgaagtggtgctgttcgggcaccagggcaaggccgagattacccaggctgagatcgaagacatcaacggtgcactgcttgcggatctgtataccgtgtggggcaattccaaccctaaaatcctgaaagatcagtaa

SEQ ID NO: 5 - protein, 387 aa, Artificial Sequence
  Description of Artificial Sequence Synthetic polypeptide
MAPPLSMTDGVAQVNTQDSNAWVEINKAAFEHNIRTLQTALAGKSQICAVLKADAYGHGIGLLMPSVIAMGVPCVGVASNEEARVVRESGFKGQLIRVRTAALSELEAALPYNMEELVGNLDFAVKASLIAEDHGRPLVVHLGLNSSGMSRNGVDMTTAQGRRDAVAITKVPNLEVRAIMTHFAVEDAADVRAGLKAFNQQAQWLMNVAQLDRSKITLHAANSFATLEVPESHLDMVRPGGALFGDTVPSHTEYKRVMQFKSHVASVNSYPKGNTVGYGRTYTLGRDSRLANITVGYSDGYRRAFTNKGIVLINGHRVPVVGKVSMNTLMVDVTDAPDVKSGDEVVLFGHQGKAEITQAEIEDINGALLADLYTVWGNSNPKILKDQ

SEQ ID NO: 6 - DNA, 2727 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaataagaaggagatatacatatggcgccacccctgtcgatgaccgacggcgtagctcaagtgaatacccaggacagcaatgcctgggtcgaaatcaataaagccgcgttcgagcacaacatacggactctgcaaaccgccctcgccggcaagtcgcagatctgcgccgtactcaaggcggatgcctatggccacggtatcggcttgttgatgccctcggtgatcgccatgggtgttccctgtgtcggtgtcgccagcaacgaagaagcccgcgtcgtgcgcgagagcggtttcaagggtcaactgatacgcgtgcgcaccgctgccctgagcgaactggaagctgcactgccgtacaacatggaagagctggtgggcaacctggacttcgcggtcaaggccagcctgattgccgaggatcacggtcgcccgctggtggtgcacctgggtctgaattccagcggcatgagccgtaacggagtggacatgaccaccgctcagggccgtcgtgatgcggtagctatcaccaaggtgccaaacctggaagtgcgggcgatcatgacccacttcgcggtcgaagatgctgccgacgtgcgtgccgggctcaaggccttcaatcagcaagcccaatggctgatgaacgtggcccagcttgatcgcagcaagatcaccctgcacgcggccaactcgttcgccacactggaggtgcccgaatcgcatctggacatggtccgccccggcggcgcgctgttcggcgacaccgtaccgtcccacaccgagtacaagcgggtcatgcagttcaagtcccacgtggcgtcggtcaacagctaccccaagggcaacaccgtcggttatggccgcacgtacaccctggggcgcgactcgcggctggccaacatcaccgtcggctactctgacggctaccgccgcgcgtttaccaataaagggattgtgctgatcaacggccatcgcgtgccagtggtgggcaaagtctcgatgaacaccctgatggtggacgtcactgacgcgccggatgtgaaaagcggcgatgaagtggtgctgttcgggcaccagggcaaggccgagattacccaggctgagatcgaagacatcaacggtgcactgcttgcggatctgtataccgtgtggggcaattccaaccctaaaatcctgaaagatcagtaa

SEQ ID NO: 7 - DNA, 1332 bp, Escherichia coli
atggtaaaggaacgtaaaaccgagttggtcgagggattccgccattcggttccctgtatcaatacccaccggggaaaaacgtttgtcatcatgctcggcggtgaagccattgagcatgagaatttctccagtatcgttaatgatatcgggttgttgcacagcctcggcatccgtctggtggtggtctatggcgcacgtccgcagatcgacgcaaatctggctgcgcatcaccacgaaccgctgtatcacaagaatatacgtgtgaccgacgccaaaacactggaactggtgaagcaggctgcgggaacattgcaactggatattactgctcgcctgtcgatgagtctcaataacacgccgctgcagggcgcgcatatcaacgtcgtcagtggcaattttattattgcccagccgctgggcgtcgatgacggcgtggattactgccatagcgggcgtatccggcggattgatgaagacgcgatccatcgtcaactggacagcggtgcaatagtgctaatggggccggtcgctgtttcagtcactggcgagagctttaacctgacctcggaagagattgccactcaactggccatcaaactgaaagctgaaaagatgattggtttttgctcttcccagggcgtcactaatgacgacggtgatattgtctccgaacttttccctaacgaagcgcaagcgcgggtagaagcccaggaagagaaaggcgattacaactccggtacggtgcgctttttgcgtggcgcagtgaaagcctgccgcagcggcgtgcgtcgctgtcatttaatcagttatcaggaagatggcgcgctgttgcaagagttgttctcacgcgacggtatcggtacgcagattgtgatggaaagcgccgagcagattcgtcgcgcaacaatcaacgatattggcggtattctggagttgattcgcccactggagcagcaaggtattctggtacgccgttctcgcgagcagctggagatggaaatcgacaaattcaccattattcagcgcgataacacgactattgcctgcgccgcgctctatccgttcccggaagagaagattggggaaatggcctgtgtggcagttcacccggattaccgcagttcatcaaggggtgaagttctgctggaacgcattgccgctcaggctaagcagagcggcttaagcaaattgtttgtgctgaccacgcgcagtattcactggttccaggaacgtggatttaccccagtggatattgatttactgcccgagagcaaaaagcagttgtacaactaccagcgtaaatccaaagtgttgatggcggatttagggtaa

SEQ ID NO: 8 - protein, 443 aa, Escherichia coli
MVKERKTELVEGFRHSVPCINTHRGKTFVIMLGGEAIEHENFSSIVNDIGLLHSLGIRLVVVYGARPQIDANLAAHHHEPLYHKNIRVTDAKTLELVKQAAGTLQLDITARLSMSLNNTPLQGAHINVVSGNFIIAQPLGVDDGVDYCHSGRIRRIDEDAIHRQLDSGAIVLMGPVAVSVTGESFNLTSEEIATQLAIKLKAEKMIGFCSSQGVTNDDGDIVSELFPNEAQARVEAQEEKGDYNSGTVRFLRGAVKACRSGVRRCHLISYQEDGALLQELFSRDGIGTQIVMESAEQIRRATINDIGGILELIRPLEQQGILVRRSREQLEMEIDKFTIIQRDNTTIACAALYPFPEEKIGEMACVAVHPDYRSSSRGEVLLERIAAQAKQSGLSKLFVLTTRSIHWFQERGFTPVDIDLLPESKKQLYNYQRKSKVLMADLG

SEQ ID NO: 9 - DNA, 1230 bp, Pseudomonas taetrolens
atgcccttctcccgtaccctgctcgccctttcccttggcatggcattgctgcaaaacccggcctttgctgcgccacccctgtcgatgaccgacggcgtagctcaagtgaatacccaggacagcaatgcctgggtcgaaatcaataaagccgcgttcgagcacaacatacggactctgcaaaccgccctcgccggcaagtcgcagatctgcgccgtactcaaggcggatgcctatggccacggtatcggcttgttgatgccctcggtgatcgccatgggtgttccctgtgtcggtgtcgccagcaacgaagaagcccgcgtcgtgcgcgagagcggtttcaagggtcaactgatacgcgtgcgcaccgctgccctgagcgaactggaagctgcactgccgtacaacatggaagagctggtgggcaacctggacttcgcggtcaaggccagcctgattgccgaggatcacggtcgcccgctggtggtgcacctgggtctgaattccagcggcatgagccgtaacggagtggacatgaccaccgctcagggccgtcgtgatgcggtagctatcaccaaggtgccaaacctggaagtgcgggcgatcatgacccacttcgcggtcgaagatgctgccgacgtgcgtgccgggctcaaggccttcaatcagcaagcccaatggctgatgaacgtggcccagcttgatcgcagcaagatcaccctgcacgcggccaactcgttcgccacactggaggtgcccgaatcgcatctggacatggtccgccccggcggcgcgctgttcggcgacaccgtaccgtcccacaccgagtacaagcgggtcatgcagttcaagtcccacgtggcgtcggtcaacagctaccccaagggcaacaccgtcggttatggccgcacgtacaccctggggcgcgactcgcggctggccaacatcaccgtcggctactctgacggctaccgccgcgcgtttaccaataaagggattgtgctgatcaacggccatcgcgtgccagtggtgggcaaagtctcgatgaacaccctgatggtggacgtcactgacgcgccggatgtgaaaagcggcgatgaagtggtgctgttcgggcaccagggcaaggccgagattacccaggctgagatcgaagacatcaacggtgcactgcttgcggatctgtataccgtgtggggcaattccaaccctaaaatcctgaaagatcagtaa

SEQ ID NO: 10 - DNA, 68 bp, Pseudomonas taetrolens
tgcccttctcccgtaccctgctcgccctttcccttggcatggcattgctgcaaaacccggcctttgct

SEQ ID NO: 11 - DNA, 1161 bp, Pseudomonas taetrolens
gcgccacccctgtcgatgaccgacggcgtagctcaagtgaatacccaggacagcaatgcctgggtcgaaatcaataaagccgcgttcgagcacaacatacggactctgcaaaccgccctcgccggcaagtcgcagatctgcgccgtactcaaggcggatgcctatggccacggtatcggcttgttgatgccctcggtgatcgccatgggtgttccctgtgtcggtgtcgccagcaacgaagaagcccgcgtcgtgcgcgagagcggtttcaagggtcaactgatacgcgtgcgcaccgctgccctgagcgaactggaagctgcactgccgtacaacatggaagagctggtgggcaacctggacttcgcggtcaaggccagcctgattgccgaggatcacggtcgcccgctggtggtgcacctgggtctgaattccagcggcatgagccgtaacggagtggacatgaccaccgctcagggccgtcgtgatgcggtagctatcaccaaggtgccaaacctggaagtgcgggcgatcatgacccacttcgcggtcgaagatgctgccgacgtgcgtgccgggctcaaggccttcaatcagcaagcccaatggctgatgaacgtggcccagcttgatcgcagcaagatcaccctgcacgcggccaactcgttcgccacactggaggtgcccgaatcgcatctggacatggtccgccccggcggcgcgctgttcggcgacaccgtaccgtcccacaccgagtacaagcgggtcatgcagttcaagtcccacgtggcgtcggtcaacagctaccccaagggcaacaccgtcggttatggccgcacgtacaccctggggcgcgactcgcggctggccaacatcaccgtcggctactctgacggctaccgccgcgcgtttaccaataaagggattgtgctgatcaacggccatcgcgtgccagtggtgggcaaagtctcgatgaacaccctgatggtggacgtcactgacgcgccggatgtgaaaagcggcgatgaagtggtgctgttcgggcaccagggcaaggccgagattacccaggctgagatcgaagacatcaacggtgcactgcttgcggatctgtataccgtgtggggcaattccaaccctaaaatcctgaaagatcagtaa

SEQ ID NO: 12 - protein, 409 aa, Pseudomonas taetrolens
MPFSRTLLALSLGMALLQNPAFAAPPLSMTDGVAQVNTQDSNAWVEINKAAFEHNIRTLQTALAGKSQICAVLKADAYGHGIGLLMPSVIAMGVPCVGVASNEEARVVRESGFKGQLIRVRTAALSELEAALPYNMEELVGNLDFAVKASLIAEDHGRPLVVHLGLNSSGMSRNGVDMTTAQGRRDAVAITKVPNLEVRAIMTHFAVEDAADVRAGLKAFNQQAQWLMNVAQLDRSKITLHAANSFATLEVPESHLDMVRPGGALFGDTVPSHTEYKRVMQFKSHVASVNSYPKGNTVGYGRTYTLGRDSRLANITVGYSDGYRRAFTNKGIVLINGHRVPVVGKVSMNTLMVDVTDAPDVKSGDEVVLFGHQGKAEITQAEIEDINGALLADLYTVWGNSNPKILKDQ

SEQ ID NO: 13 - protein, 23 aa, Pseudomonas taetrolens
MPFSRTLLALSLGMALLQNPAFA

SEQ ID NO: 14 - protein, 387 aa, Pseudomonas taetrolens
AAPPLSMTDGVAQVNTQDSNAWVEINKAAFEHNIRTLQTALAGKSQICAVLKADAYGHGIGLLMPSVIAMGVPCVGVASNEEARVVRESGFKGQLIRVRTAALSELEAALPYNMEELVGNLDFAVKASLIAEDHGRPLVVHLGLNSSGMSRNGVDMTTAQGRRDAVAITKVPNLEVRAIMTHFAVEDAADVRAGLKAFNQQAQWLMNVAQLDRSKITLHAANSFATLEVPESHLDMVRPGGALFGDTVPSHTEYKRVMQFKSHVASVNSYPKGNTVGYGRTYTLGRDSRLANITVGYSDGYRRAFTNKGIVLINGHRVPVVGKVSMNTLMVDVTDAPDVKSGDEVVLFGHQGKAEITQAEIEDINGALLADLYTVWGNSNPKILKDQ

SEQ ID NO: 15 - DNA, 18 bp, Artificial Sequence
  Description of Artificial Sequence Synthetic oligonucleotide
  modified_base (2)..(2): a, c, t, g, unknown or other
  modified_base (17)..(17): a, c, t, g, unknown or other
wntgaatwwwwattcanw

(19) United States
(12) Patent Application Publication
(10) Pub. No.: US 2021/0130806 A1
(43) Pub. Date: May 6, 2021

(54) ENGINEERED BACTERIA EXPRESSING RACEMASE FOR TREATING DISEASES ASSOCIATED WITH HYPERAMMONEMIA

(71) Applicant: Synlogic Operating Company, Inc., Cambridge, MA (US)

(72) Inventors: Yves Millet, Newton, MA (US); Vincent M. Isabella, Medford, MA (US); Jonathan W. Kotula, Berkeley, CA (US); Paul F. Miller, Salem, CT (US); Dean Falb, Sherborn, MA (US)

(21) Appl. No.: 16/760,978

(22) PCT Filed: Nov. 2, 2018
(86) PCT No.: PCT/US2018/058989

§ 371 Date: May 1, 2020

Related U.S. Application Data
(60) Provisional application No. 62/640,887, filed on Mar. 9, 2018.
(60) Provisional application No. 62/581,498, filed on Nov. 3, 2017.

(51) Int. Cl. C12N 9/90; C12N 1/20; A61K 35/74; A61K 38/52; A61P 3/00; G01N 33/68

(52) U.S. Cl. CPC C12N 9/90; C12N 1/20; C12Y 501/01009; G01N 2800/52; A61K 38/52; A61P 3/00; G01N 33/68; A61K 35/74

(57) ABSTRACT

Disclosed herein are bacteria engineered to treat diseases associated with hyperammonemia and methods of use thereof. Specifically, the bacteria are engineered to comprise a racemase to enable the detection of non-naturally occurring metabolites as an indication that the engineered bacteria are effectively converting ammonia.

### RELATED APPLICATION

This application claims priority to U.S. Provisional Application No. 62/581,498, filed on Nov. 3, 2017 and U.S. Provisional Application No. 62/640,887, filed on Mar. 9, 2018, the entire contents of each of which are expressly incorporated by reference.

### SEQUENCE LISTING

The instant application contains a Sequence Listing which has been submitted electronically in ASCII format and is hereby incorporated by reference in its entirety. Said ASCII copy, created on Nov. 1, 2018, is named 126046-04020_SL.txt and is 29,070 bytes in size.

### BACKGROUND

Ammonia is highly toxic and generated during metabolism in all organs (Walker, 2012). In mammals, the healthy liver protects the body from ammonia by converting ammonia to non-toxic molecules, e.g., urea or glutamine, and preventing excess amounts of ammonia from entering the systemic circulation. Hyperammonemia is characterized by the decreased detoxification and/or increased production of ammonia. In mammals, the urea cycle detoxifies ammonia by enzymatically converting ammonia into urea, which is then removed in the urine. Decreased ammonia detoxification may be caused by urea cycle disorders (UCDs) in which urea cycle enzymes are defective, such as argininosuccinic aciduria, arginase deficiency, carbamoylphosphate synthetase deficiency, citrullinemia, N-acetylglutamate synthetase deficiency, and ornithine transcarbamylase deficiency (Häberle et al., 2012). The National Urea Cycle Disorders Foundation estimates that the prevalence of UCDs is 1 in 8,500 births. In addition, several non-UCD disorders, such as hepatic encephalopathy, portosystemic shunting, and organic acid disorders, can also cause hyperammonemia. Hyperammonemia can produce neurological manifestations, e.g., seizures, ataxia, stroke-like lesions, coma, psychosis, vision loss, acute encephalopathy, cerebral edema, as well as vomiting, respiratory alkalosis, hypothermia, or death (Häberle et al., 2012; Häberle et al., 2013).

Ammonia is also a source of nitrogen for amino acids, which are synthesized by various biosynthesis pathways. For example, arginine biosynthesis converts glutamate, which comprises one nitrogen atom, to arginine, which comprises four nitrogen atoms. Intermediate metabolites formed in the arginine biosynthesis pathway, such as citrulline, also incorporate nitrogen. Thus, enhancement of arginine biosynthesis may be used to incorporate excess nitrogen in the body into non-toxic molecules in order to modulate or treat conditions associated with hyperammonemia. Likewise, histidine biosynthesis, methionine biosynthesis, lysine biosynthesis, asparagine biosynthesis, glutamine biosynthesis, and tryptophan biosynthesis are also capable of incorporating excess nitrogen, and enhancement of those pathways may be used to modulate or treat conditions associated with hyperammonemia.

Current therapies for hyperammonemia and UCDs aim to reduce ammonia excess, but are widely regarded as suboptimal (Nagamani et al., 2012; Hoffmann et al., 2013; Torres-Vega et al., 2014). Most UCD patients require substantially modified diets consisting of protein restriction. However, a low-protein diet must be carefully monitored; when protein intake is too restrictive, the body breaks down muscle and consequently produces ammonia. In addition, many patients require supplementation with ammonia scavenging drugs, such as sodium phenylbutyrate, sodium benzoate, and glycerol phenylbutyrate, and one or more of these drugs must be administered three to four times per day (Leonard, 2006; Diaz et al., 2013). Side effects of these drugs include nausea, vomiting, irritability, anorexia, and menstrual disturbance in females (Leonard, 2006). In children, the delivery of food and medication may require a gastrostomy tube surgically implanted in the stomach or a nasogastric tube manually inserted through the nose into the stomach. When these treatment options fail, a liver transplant may be required (National Urea Cycle Disorders Foundation). Thus, there is significant unmet need for effective, reliable, and/or long-term treatment for disorders associated with hyperammonemia, including urea cycle disorders.

The liver plays a central role in amino acid metabolism and protein synthesis and breakdown, as well as in several detoxification processes, notably those of end-products of intestinal metabolism, like ammonia. Liver dysfunction, resulting in hyperammonemia, may cause hepatic encephalopathy (HE), which disorder encompasses a spectrum of potentially reversible neuropsychiatric abnormalities observed in patients with liver dysfunction (after exclusion of unrelated neurologic and/or metabolic abnormalities). In HE, severe liver failure (e.g., cirrhosis) and/or portosystemic shunting of blood around the liver permit elevated arterial levels of ammonia to permeate the blood-brain barrier (Williams, 2006), resulting in altered brain function.

Ammonia accumulation in the brain leads to cognitive and motor disturbances, reduced cerebral perfusion, as well as oxidative stress-mediated injury to astrocytes, the brain cells capable of metabolizing ammonia. There is evidence to suggest that excess ammonia in the brain disrupts neurotransmission by altering levels of the predominant inhibitory neurotransmitter, γ-aminobutyric acid (GABA) (Ahboucha and Butterworth, 2004). Elevated cerebral manganese concentrations and manganese deposition have also been reported in the basal ganglia of cirrhosis patients, and are suspected to contribute to the clinical presentation of HE (Cash et al., 2010; Rivera-Mancía et al., 2012). General neurological manifestations of hyperammonemia include seizures, ataxia, stroke-like lesions, Parkinsonian symptoms (such as tremors), coma, psychosis, vision loss, acute encephalopathy, cerebral edema, as well as vomiting, respiratory alkalosis, hypothermia, or death (Häberle et al., 2012; Häberle et al., 2013).

Ammonia dysmetabolism cannot solely explain all the neurological changes that are seen in patients with HE. Sepsis is a well-known precipitating factor for HE. The systemic inflammatory response syndrome (SIRS) results from the release and circulation of proinflammatory cytokines and mediators. In patients with cirrhosis, SIRS may exacerbate the symptoms of HE, both in patients with minimal and overt HE in a process likely mediated by tumor necrosis factor (TNF) and interleukin-6 (IL6). Notably, enhanced production of reactive nitrogen species (RNS) and reactive oxygen species (ROS) occurs in cultured astrocytes that are exposed to ammonia, inflammatory cytokines, hyponatremia or benzodiazepines.

Hyperammonemia is also a prominent feature of Huntington's disease, an autosomal dominant disorder characterized by intranuclear/cytoplasmic aggregates and cell death in the brain (Chen et al., 2015; Chiang et al., 2007). In fact, hyperammonemia is a feature of several other disorders, as discussed herein, all of which can be treated by reducing the levels of ammonia.

Current therapies for hepatic encephalopathy, Huntington's disease, and other diseases and disorders associated with excess ammonia levels, are insufficient (Cash et al., 2010; Cordoba and Mínguez, 2008; Shannon and Fraint, 2015). In Huntington's disease, the side effects of antipsychotic drugs (e.g., haloperidol, risperidone, quetiapine) and drugs administered to suppress involuntary movements (e.g., tetrabenazine, amantadine, levetiracetam, clonazepam) may worsen muscle rigidity and cognitive decline in patients (Mayo Clinic). Antibiotics directed to urease-producing bacteria were shown to have severe secondary effects, such as nephrotoxicity, especially if administered for long periods (Blanc et al., 1992; Berk and Chalmers, 1970). Protein restriction is also no longer a mainstay therapy, as it can favor protein degradation and poor nutritional status, and has been associated with increased mortality (Kondrup and Müller, 1997; Vaqero et al., 2003). Protein restriction is only appropriate for one third of cirrhotic patients with HE (Nguyen and Morgan, 2014). Thus, there is significant unmet need for effective, reliable, and/or long-term treatment for hepatic encephalopathy and Huntington's disease.

### SUMMARY

The disclosure provides genetically engineered bacteria that are capable of reducing excess ammonia and converting ammonia and/or nitrogen into alternate byproducts. In certain embodiments, the genetically engineered bacteria reduce excess ammonia and convert ammonia and/or nitrogen into alternate byproducts. In certain embodiments, the genetically engineered bacteria are non-pathogenic and may be introduced into the gut in order to reduce toxic ammonia. As much as 70% of excess ammonia in a hyperammonemic patient accumulates in the gastrointestinal tract. In some embodiments, the engineered bacteria are further capable of producing butyrate, or capable of improved butyrate production.

Another aspect of the invention provides methods for selecting or targeting genetically engineered bacteria based on increased levels of ammonia and/or nitrogen consumption, or production of a non-toxic byproduct, e.g., arginine or citrulline. In some embodiments, the engineered bacteria of the invention further express a racemase which is capable of converting, e.g., L-arginine to D-arginine. D-arginine and/or L-arginine can then be measured in the urine and/or feces of patients which are administered the engineered bacteria as an indication that the engineered bacteria are effectively converting ammonia to L-arginine and then to D-arginine.

The invention also provides pharmaceutical compositions comprising the genetically engineered bacteria, and methods of modulating and treating disorders associated with hyperammonemia, e.g., urea cycle disorders and hepatic encephalopathy.

The invention also provides pharmaceutical compositions comprising the genetically engineered bacteria, and methods of modulating and treating disorders associated with excess ammonia, including, for example, hepatic encephalopathy and Huntington's disease. The invention also provides pharmaceutical compositions, biomarkers, and methods for the detection of active ammonia reducing bacteria in vivo in a subject.

In some embodiments, the genetically engineered bacteria comprise one or more gene(s) or gene cassette(s) or circuit(s), containing one or more native or non-native component(s), which mediate one or more mechanisms of action. Additionally, one or more endogenous genes or regulatory regions within the bacterial chromosome may be mutated or deleted. The genetically engineered bacteria harbor these genes or gene cassettes or circuits on a plasmid or, alternatively, the genes/gene cassettes have been inserted into the chromosome at certain regions, where they do not interfere with essential gene expression.

In some embodiments, the genetically engineered bacteria are capable of converting L-arginine into D-arginine. In some embodiments, the genetically engineered bacteria comprise one or more genes encoding one or more arginine racemases for the conversion of L-arginine into D-arginine.

In some embodiments, the genetically engineered bacteria comprise one or more gene sequences encoding a feedback resistant N-acetylglutamate synthetase (ArgA), and further comprise a mutation or deletion in the endogenous feedback repressor of arginine synthesis ArgR. In some embodiments, the genetically engineered bacteria comprise a deletion or mutation in the ThyA gene.

These gene(s)/gene cassette(s) may be under the control of constitutive or inducible promoters. Exemplary inducible promoters described herein include oxygen level-dependent promoters (e.g., FNR-inducible promoter), promoters induced by HE-specific molecules or metabolites indicative of liver damage (e.g., bilirubin), promoters induced by inflammation or an inflammatory response (RNS, ROS promoters), and promoters induced by a metabolite that may or may not be naturally present (e.g., can be exogenously added) in the gut, e.g., arabinose and tetracycline.

In addition, the engineered bacteria may further comprise one or more of more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

In one aspect, disclosed herein is an engineered bacterium capable of reducing excess ammonia or capable of converting ammonia and/or nitrogen into an alternate byproduct, wherein the bacterium comprises a racemase. In one embodiment, the alternate byproduct is L-arginine.

In one embodiment, the racemase is an amino acid racemase. In one embodiment, the amino acid racemase is an arginine racemase. In one embodiment, the racemase is a d1-23 racemase. In one embodiment, the racemase is an ArR racemase. In one embodiment, the racemase is from Pseudomonas taetrolens. In one embodiment, the racemase is selected from the group consisting of EC 5.1.1.1 (alanine racemase), EC 5.1.1.2 (methionine racemase), EC 5.1.1.3 (glutamine racemase), EC 5.1.1.4 (proline racemase), EC 5.1.1.5 (lysine racemase), EC 5.1.1.6 (threonine racemase), EC 5.1.1.7 (diaminopimelate epimerase), EC 5.1.1.8 (4-hydroxyproline epimerase), EC 5.1.1.9 (arginine racemase), EC 5.1.1.10 (amino acid racemase), EC 5.1.1.11 (phenylalanine racemase), EC 5.1.1.12 (ornithine racemase), EC 5.1.1.13 (aspartate racemase), EC 5.1.1.14 (nocardicin-A epimerase), EC 5.1.1.15 (2-aminohexano-6-lactam racemase), EC 5.1.1.16 (protein-serine racemase), EC 5.1.1.17 (isopenicillin-N racemase), and EC 5.1.1.18 (serine racemase).

In one embodiment, the racemase does not comprise a signal peptide. In one embodiment, the racemase does comprise a signal peptide.

In one embodiment, the racemase comprises a sequence that is at least 90% identical to SEQ ID NO:5, SEQ ID NO:12, or SEQ ID NO:14. In one embodiment, the racemase is encoded by a sequence that is at least 90% identical to SEQ ID NO:4, SEQ ID NO:9, or SEQ ID NO:11.

In one embodiment, the engineered bacterium comprises a modification to lack a functional ArgR. In one embodiment, ArgR is mutated. In one embodiment, argR is deleted.

In one embodiment, the bacterium is an auxotroph in a gene that is complemented when the bacterium is present in a mammalian gut. In one embodiment, the bacterium is a thyA auxotroph.

In one embodiment, the bacterium further comprises an arginine feedback resistant N-acetylglutamate synthetase (ArgA^{fbr}).

In one embodiment, the bacterium comprises argE, argC, argB, argH, argD, argF, argI, argG, carA, carB, argA^{fbr}, and a modification to lack a functional ArgR. In one embodiment, ArgR is mutated. In one embodiment, argR is deleted. In one embodiment, the argA^{fbr} is under the control of an FNR promoter. In one embodiment, the bacterium further comprises ter, thiA1, hbd, crt2, ptb, and buk. In one embodiment, the ter, thiA1, hbd, crt2, ptb, and buk genes are under the control of an FNR promoter.

In one embodiment, the bacterium is engineered to express ArgE, ArgC, ArgB, ArgH, ArgD, ArgF, ArgI, ArgG, CarA, CarB, and ArgA^{fbr}, and a modification to lack a functional ArgG. In one embodiment, the bacterium is engineered to further express Ter, ThiA1, Hbd, Crt2, Ptb, and Buk.

In another aspect, disclosed herein is a pharmaceutical composition comprising the engineered bacterium.

In another aspect, disclosed herein is a method of treating a subject, the method comprising administering an engineered bacterium, or a pharmaceutical composition to the subject, thereby treating the subject.

In another aspect, disclosed herein is a method of decreasing ammonia levels in a subject, the method comprising administering the engineered bacterium, or the pharmaceutical composition, to the subject, thereby decreasing ammonia levels in the subject.

In one embodiment, the method further comprises collecting a urine sample from the subject and measuring the level of D-arginine and/or L-arginine in the sample. In one embodiment, the method further comprises collecting a feces sample from the subject and measuring the level of D-arginine and/or L-arginine in the sample.

In another aspect, disclosed herein is a method of monitoring the treatment of a subject and who has previously been administered the engineered bacterium, the method comprising measuring levels of D-arginine and/or L-arginine in the urine of the subject, thereby monitoring the treatment of the subject. In another aspect, disclosed herein is a method of monitoring the treatment of a subject and who has previously been administered the engineered bacterium, the method comprising measuring levels of D-arginine and/or L-arginine in the feces of the subject, thereby monitoring the treatment of the subject.

In another aspect, disclosed herein is the use of the engineered bacterium as an indicator of the ability of the bacterium to reduce excess ammonia or convert ammonia and/or nitrogen into an alternate byproduct, wherein the use comprises measuring levels of D-arginine and/or L-arginine in the urine and/or feces of a subject who has previously been administered the engineered bacterium.

In one embodiment, an increased level of D-arginine in the urine of the subject as compared to a control indicates that the engineered bacterium is reducing excess ammonia and/or converting ammonia and/or nitrogen into an alternate byproduct. In one embodiment, an increased level of D-arginine in the feces of the subject as compared to a control indicates that the engineered bacterium is reducing excess ammonia and/or converting ammonia and/or nitrogen into an alternate byproduct. In one embodiment, the control is a level of D-arginine in the subject prior to administration of the engineered bacterium. In one embodiment, the control is a level of D-arginine from a population of subjects not treated with the engineered bacterium.

In one embodiment, the level of D-arginine in the urine and/or feces of the subject is increased at least 1.5 fold, or about 1.5 fold, as compared to the control. In one embodiment, the level of D-arginine in the urine and/or feces of the subject is increased at least 2 fold, or about 2 fold, as compared to the control. In one embodiment, the level of D-arginine in the urine and/or feces of the subject is increased at least 6 fold, or about 6 fold, as compared to the control.

In one embodiment, the subject has a urea cycle disorder (UCD).

### BRIEF DESCRIPTION OF THE FIGURES

FIG. 1 depicts a bar graph showing levels of in vitro D and L arginine production, comparing E. coli Nissle (SYN94), an ammonia consuming (arginine producing) strain (SYN-UCD824) comprising a deletion in argR, and tetracycline inducible argA^{fbr} integrated into the chromosome at the malEK site, and an ammonia consuming (arginine producing) strain derived from SYN-UCD824 which further comprises a tet inducible arginine racemase from Pseudomonas taetrolens (arR) on a low copy plasmid (SYN-UCD3230). Results show that that the racemase, expressed on a low-copy plasmid (pSC101), is functional.

FIG. 2 depicts a bar graph showing levels of in vitro D and L arginine production, comparing E. coli Nissle (SYN94), an ammonia consuming (arginine producing) strain (SYN-UCD824) comprising a deletion in argR, and tetracycline inducible argA^{fbr} integrated into the chromosome at the malEK site, and an ammonia consuming (arginine producing) strain derived from SYN-UCD824 which further comprises on a low copy plasmid a tet inducible arginine racemase from Pseudomonas taetrolens, which has a truncation in the first 23 AA of the polypeptide (arRΔ1-69), removing the signal sequence for export from the cell into the periplasm (SYN-UCD3331). Results show that truncating the first 23 AA (the signal peptide for export to periplasm) does not impair racemase activity.

FIG. 3 depicts a bar graph showing levels of in vitro D and L arginine production, comparing three strains with integrated circuitry. SYN-UCD305 comprises ΔargR, feedback resistant argA under the control of an FNR promoter, and an auxotrophy (ΔthyA). SYN-UCD3649 comprises arRΔ1-69 under the control of an FNR promoter, and ΔargR and ΔthyA. SYN-UCD3650 comprises argA^{fbr} and arRΔ1-69 arranged in tandem, both under the control of an FNR promoter, ΔargR, and ΔthyA. Results show the activity of the truncated racemase integrated downstream of argA^{fbr} and under control of the FNR promoter.

FIG. 4A and FIG. 4B depict graphs showing measurement of urinary D-arginine (FIG. 4A) and plasma D-arginine (FIG. 4B) over time in mice gavaged with SYN-UCD3645 (malEK:P_{fnrs}-arRΔ1-69) or SYN-UCD3650. Wild-type C57B6 mice were dosed orally with 1e10 CFUs and placed in metabolic cages for collection of urine over 8 hours. Urine was analyzed for biomarkers including D-arg and L-arg. Results show the racemase is active in vivo and that production of D-arginine through racemase activity is useful as a biomarker for the activity of the ammonia consuming circuitry (more D-arg is excreted in mice gavaged with the strain engineered to produce L-arg than in mice gavaged with the Nissle expressing the racemase alone).

FIG. 5A and FIG. 5B depict graphs showing area under the curve (AUC) of plasma D-arginine (FIG. 5A) and total D-arginine in urine (FIG. 5B) of cynomolgus monkeys, which were fasted overnight and dosed with 10¹² cells of SYN-UCD3650 (malEK:P_{fnrS}-argA^{fbr}-arR^{Δ1-69}; ΔargR; ΔthyA) or SYN-UCD3645 (malEK:P_{fnrS}-arR^{Δ1-69}). The level of D-arginine in blood and urine samples were analyzed by LC-MS/MS using a chiral column allowing the separation of L and D-arginine enantiomers.

### DESCRIPTION OF EMBODIMENTS

The invention includes genetically engineered bacteria, pharmaceutical compositions thereof, and methods of modulating or treating disorders associated with hyperammonemia, e.g., urea cycle disorders, hepatic encephalopathy and other disorders associated with excess ammonia or elevated ammonia levels. The genetically engineered bacteria are capable of reducing excess ammonia, particularly under certain environmental conditions, such as those in the mammalian gut. In some embodiments, the genetically engineered bacteria reduce excess ammonia by incorporating excess nitrogen in the body into non-toxic molecules, e.g., arginine, citrulline, methionine, histidine, lysine, asparagine, glutamine, or tryptophan, or pyrimidines.

In any of the described embodiments, the engineered bacteria may further comprise one or more of more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, e.g., ammonia, such any of the transporters described herein or otherwise known in the art, (5) one or more secretion circuits, such as any of the secretion circuits described herein and otherwise known in the art, and (6) combinations of one or more of such additional circuits.

Furthermore, the engineered bacteria may further be capable of producing butyrate. Metabolic pathways for butyrate production are well known in the art and are described, for example in WO17/123418, published on Jul. 20, 2017, the entire contents of which are expressly incorporated herein by reference. For example, a butyrate producing cassette may comprise at least the following genes: ter, thiA1, hbd, crt2, pbt, and buk.

“Hyperammonemia,” “hyperammonemic,” or “excess ammonia” is used to refer to increased concentrations of ammonia in the body. Hyperammonemia is caused by decreased detoxification and/or increased production of ammonia. Decreased detoxification may result from urea cycle disorders (UCDs), such as argininosuccinic aciduria, arginase deficiency, carbamoylphosphate synthetase deficiency, citrullinemia, N-acetylglutamate synthetase deficiency, and ornithine transcarbamylase deficiency; or from bypass of the liver, e.g., open ductus hepaticus; and/or deficiencies in glutamine synthetase (Hoffman et al., 2013; Häberle et al., 2013). Decreased detoxification may also result from liver disorders such as hepatic encephalopathy, acute liver failure, or chronic liver failure; and neurodegenerative disorders such as Huntington's disease (Chen et al., 2015; Chiang et al., 2007). Increased production of ammonia may result from infections, drugs, neurogenic bladder, and intestinal bacterial overgrowth (Häberle et al., 2013). Other disorders and conditions associated with hyperammonemia include, but are not limited to, liver disorders such as hepatic encephalopathy, acute liver failure, or chronic liver failure; organic acid disorders; isovaleric aciduria; 3-methylcrotonylglycinuria; methylmalonic acidemia; propionic aciduria; fatty acid oxidation defects; carnitine cycle defects; carnitine deficiency; β-oxidation deficiency; lysinuric protein intolerance; pyrroline-5-carboxylate synthetase deficiency; pyruvate carboxylase deficiency; ornithine aminotransferase deficiency; carbonic anhydrase deficiency; hyperinsulinism-hyperammonemia syndrome; mitochondrial disorders; valproate therapy; asparaginase therapy; total parenteral nutrition; cystoscopy with glycine-containing solutions; post-lung/bone marrow transplantation; portosystemic shunting; urinary tract infections; ureter dilation; multiple myeloma; and chemotherapy (Hoffman et al., 2013; Häberle et al., 2013; Pham et al., 2013; Lazier et al., 2014). In healthy subjects, plasma ammonia concentrations are typically less than about 50 μmon (Leonard, 2006). In some embodiments, a diagnostic signal of hyperammonemia is a plasma ammonia concentration of at least about 50 μmon, at least about 80 μmol/L, at least about 150 μmol/L, at least about 180 μmol/L, or at least about 200 μmol/L (Leonard, 2006; Hoffman et al., 2013; Häberle et al., 2013).

“Ammonia” is used to refer to gaseous ammonia (NH₃), ionic ammonia (NH₄⁺), or a mixture thereof. In bodily fluids, gaseous ammonia and ionic ammonium exist in equilibrium: NH₃+H⁺↔NH₄⁺

Some clinical laboratory tests analyze total ammonia (NH₃+NH₄⁺) (Walker, 2012). In any embodiment of the invention, unless otherwise indicated, “ammonia” may refer to gaseous ammonia, ionic ammonia, and/or total ammonia.

“Detoxification” of ammonia is used to refer to the process or processes, natural or synthetic, by which toxic ammonia is removed and/or converted into one or more non-toxic molecules, including but not limited to: arginine, citrulline, methionine, histidine, lysine, asparagine, glutamine, tryptophan, or urea. The urea cycle, for example, enzymatically converts ammonia into urea for removal from the body in the urine. Because ammonia is a source of nitrogen for many amino acids, which are synthesized via numerous biochemical pathways, enhancement of one or more of those amino acid biosynthesis pathways may be used to incorporate excess nitrogen into non-toxic molecules. For example, arginine biosynthesis converts glutamate, which comprises one nitrogen atom, to arginine, which comprises four nitrogen atoms, thereby incorporating excess nitrogen into non-toxic molecules. In humans, arginine is not reabsorbed from the large intestine, and as a result, excess arginine in the large intestine is not considered to be harmful. Likewise, citrulline is not reabsorbed from the large intestine, and as a result, excess citrulline in the large intestine is not considered to be harmful. Arginine biosynthesis may also be modified to produce citrulline as an end product; citrulline comprises three nitrogen atoms and thus the modified pathway is also capable of incorporating excess nitrogen into non-toxic molecules.

“Arginine regulon,” “arginine biosynthesis regulon,” and “arg regulon” are used interchangeably to refer to the collection of operons in a given bacterial species that comprise the genes encoding the enzymes responsible for converting glutamate to arginine and/or intermediate metabolites, e.g., citrulline, in the arginine biosynthesis pathway. The arginine regulon also comprises operators, promoters, ARG boxes, and/or regulatory regions associated with those operons.

The arginine regulon includes, but is not limited to, the operons encoding the arginine biosynthesis enzymes N-acetylglutamate synthetase, N-acetylglutamate kinase, N-acetylglutamylphosphate reductase, acetylornithine aminotransferase, N-acetylornithinase, ornithine transcarbamylase, argininosuccinate synthase, argininosuccinate lyase, carbamoylphosphate synthase, operators thereof, promoters thereof, ARG boxes thereof, and/or regulatory regions thereof. In some embodiments, the arginine regulon comprises an operon encoding ornithine acetyltransferase and associated operators, promoters, ARG boxes, and/or regulatory regions, either in addition to or in lieu of N-acetylglutamate synthetase and/or N-acetylornithinase. In some embodiments, one or more operons or genes of the arginine regulon may be present on a plasmid in the bacterium. In some embodiments, a bacterium may comprise multiple copies of any gene or operon in the arginine regulon, wherein one or more copies may be mutated or otherwise altered as described herein.

One gene may encode one enzyme, e.g., N-acetylglutamate synthetase (argA). Two or more genes may encode distinct subunits of one enzyme, e.g., subunit A and subunit B of carbamoylphosphate synthase (carA and carB). In some bacteria, two or more genes may each independently encode the same enzyme, e.g., ornithine transcarbamylase (argF and argI). In some bacteria, the arginine regulon includes, but is not limited to, argA, encoding N-acetylglutamate synthetase; argB, encoding N-acetylglutamate kinase; argC, encoding N-acetylglutamylphosphate reductase; argD, encoding acetylornithine aminotransferase; argE, encoding N-acetylornithinase; argG, encoding argininosuccinate synthase; argH, encoding argininosuccinate lyase; one or both of argF and argI, each of which independently encodes ornithine transcarbamylase; carA, encoding the small subunit of carbamoylphosphate synthase; carB, encoding the large subunit of carbamoylphosphate synthase; operons thereof; operators thereof; promoters thereof; ARG boxes thereof; and/or regulatory regions thereof. In some embodiments, the arginine regulon comprises argJ, encoding ornithine acetyltransferase (either in addition to or in lieu of N-acetylglutamate synthetase and/or N-acetylornithinase), operons thereof, operators thereof, promoters thereof, ARG boxes thereof, and/or regulatory regions thereof.

“Arginine operon,” “arginine biosynthesis operon,” and “arg operon” are used interchangeably to refer to a cluster of one or more of the genes encoding arginine biosynthesis enzymes under the control of a shared regulatory region comprising at least one promoter and at least one ARG box. In some embodiments, the one or more genes are co-transcribed and/or co-translated. Any combination of the genes encoding the enzymes responsible for arginine biosynthesis may be organized, naturally or synthetically, into an operon. For example, in B. subtilis, the genes encoding N-acetylglutamylphosphate reductase, N-acetylglutamate kinase, N-acetylornithinase, N-acetylglutamate kinase, acetylornithine aminotransferase, carbamoylphosphate synthase, and ornithine transcarbamylase are organized in a single operon, argCAEBD-carAB-argF (see, e.g., Table 2), under the control of a shared regulatory region comprising a promoter and ARG boxes. In E. coli K12 and Nissle, the genes encoding N-acetylornithinase, N-acetylglutamylphosphate reductase, N-acetylglutamate kinase, and argininosuccinate lyase are organized in two bipolar operons, argECBH. The operons encoding the enzymes responsible for arginine biosynthesis may be distributed at different loci across the chromosome. In unmodified bacteria, each operon may be repressed by arginine via ArgR. In some embodiments, arginine and/or intermediate byproduct production may be altered in the genetically engineered bacteria of the invention by modifying the expression of the enzymes encoded by the arginine biosynthesis operons as provided herein. Each arginine operon may be present on a plasmid or bacterial chromosome. In addition, multiple copies of any arginine operon, or a gene or regulatory region within an arginine operon, may be present in the bacterium, wherein one or more copies of the operon or gene or regulatory region may be mutated or otherwise altered as described herein. In some embodiments, the genetically engineered bacteria are engineered to comprise multiple copies of the same product (e.g., operon or gene or regulatory region) to enhance copy number or to comprise multiple different components of an operon performing multiple different functions.

“ARG box consensus sequence” refers to an ARG box nucleic acid sequence, the nucleic acids of which are known to occur with high frequency in one or more of the regulatory regions of argR, argA, argB, argC, argD, argE, argF, argG, argH, argI, argJ, carA, and/or carB. As described above, each arg operon comprises a regulatory region comprising at least one 18-nucleotide imperfect palindromic sequence, called an ARG box, that overlaps with the promoter and to which the repressor protein binds (Tian et al., 1992). The nucleotide sequences of the ARG boxes may vary for each operon, and the consensus ARG box sequence is ^{A}/_{T} nTGAAT ^{A}/_{T} ^{A}/_{T} ^{T}/_{A} T/A ATTCAn ^{T}/_{A} (SEQ ID NO: 15) (Maas, 1994). The arginine repressor binds to one or more ARG boxes to actively inhibit the transcription of the arginine biosynthesis enzyme(s) that are operably linked to that one or more ARG boxes.

“Mutant arginine regulon” or “mutated arginine regulon” is used to refer to an arginine regulon comprising one or more nucleic acid mutations that reduce or eliminate arginine-mediated repression of each of the operons that encode the enzymes responsible for converting glutamate to arginine and/or an intermediate byproduct, e.g., citrulline, in the arginine biosynthesis pathway, such that the mutant arginine regulon produces more arginine and/or intermediate byproduct than an unmodified regulon from the same bacterial subtype under the same conditions. In some embodiments, the genetically engineered bacteria comprise an arginine feedback resistant N-acetylglutamate synthase mutant, e.g., argA^{fbr}, and a mutant arginine regulon comprising one or more nucleic acid mutations in at least one ARG box for one or more of the operons that encode the arginine biosynthesis enzymes N-acetylglutamate kinase, N-acetylglutamylphosphate reductase, acetylornithine aminotransferase, N-acetylornithinase, ornithine transcarbamylase, argininosuccinate synthase, argininosuccinate lyase, and carbamoylphosphate synthase, thereby derepressing the regulon and enhancing arginine and/or intermediate byproduct biosynthesis. In some embodiments, the genetically engineered bacteria comprise a mutant arginine repressor comprising one or more nucleic acid mutations such that arginine repressor function is decreased or inactive, or the genetically engineered bacteria do not have an arginine repressor (e.g., the arginine repressor gene has been deleted), resulting in derepression of the regulon and enhancement of arginine and/or intermediate byproduct biosynthesis. In some embodiments, the genetically engineered bacteria comprise an arginine feedback resistant N-acetylglutamate synthase mutant, e.g., argA^{fbr}, a mutant arginine regulon comprising one or more nucleic acid mutations in at least one ARG box for each of the operons that encode the arginine biosynthesis enzymes, and/or a mutant or deleted arginine repressor. In some embodiments, the genetically engineered bacteria comprise an arginine feedback resistant N-acetylglutamate synthase mutant, e.g., argA^{fbr} and a mutant arginine regulon comprising one or more nucleic acid mutations in at least one ARG box for each of the operons that encode the arginine biosynthesis enzymes. In some embodiments, the genetically engineered bacteria comprise an arginine feedback resistant N-acetylglutamate synthase mutant, e.g., argA^{fbr} and a mutant or deleted arginine repressor. In some embodiments, the mutant arginine regulon comprises an operon encoding wild-type N-acetylglutamate synthetase and one or more nucleic acid mutations in at least one ARG box for said operon. In some embodiments, the mutant arginine regulon comprises an operon encoding wild-type N-acetylglutamate synthetase and mutant or deleted arginine repressor. In some embodiments, the mutant arginine regulon comprises an operon encoding ornithine acetyltransferase (either in addition to or in lieu of N-acetylglutamate synthetase and/or N-acetylornithinase) and one or more nucleic acid mutations in at least one ARG box for said operon.

The ARG boxes overlap with the promoter in the regulatory region of each arginine biosynthesis operon. In the mutant arginine regulon, the regulatory region of one or more arginine biosynthesis operons is sufficiently mutated to disrupt the palindromic ARG box sequence and reduce ArgR binding, but still comprises sufficiently high homology to the promoter of the non-mutant regulatory region to be recognized as the native operon-specific promoter. The operon comprises at least one nucleic acid mutation in at least one ARG box such that ArgR binding to the ARG box and to the regulatory region of the operon is reduced or eliminated. In some embodiments, bases that are protected from DNA methylation and bases that are protected from hydroxyl radical attack during ArgR binding are the primary targets for mutations to disrupt ArgR binding (see, e.g., Table 3). The promoter of the mutated regulatory region retains sufficiently high homology to the promoter of the non-mutant regulatory region such that RNA polymerase binds to it with sufficient affinity to promote transcription of the operably linked arginine biosynthesis enzyme(s). In some embodiments, the G/C:A/T ratio of the promoter of the mutant differs by no more than 10% from the G/C:A/T ratio of the wild-type promoter.

In some embodiments, more than one ARG box may be present in a single operon. In one aspect of these embodiments, at least one of the ARG boxes in an operon is altered to produce the requisite reduced ArgR binding to the regulatory region of the operon. In an alternate aspect of these embodiments, each of the ARG boxes in an operon is altered to produce the requisite reduced ArgR binding to the regulatory region of the operon.

“Reduced” ArgR binding is used to refer to a reduction in repressor binding to an ARG box in an operon or a reduction in the total repressor binding to the regulatory region of said operon, as compared to repressor binding to an unmodified ARG box and regulatory region in bacteria of the same subtype under the same conditions. In some embodiments, ArgR binding to a mutant ARG box and regulatory region of an operon is at least about 50% lower, at least about 60% lower, at least about 70% lower, at least about 80% lower, at least about 90% lower, or at least about 95% lower than ArgR binding to an unmodified ARG box and regulatory region in bacteria of the same subtype under the same conditions. In some embodiments, reduced ArgR binding to a mutant ARG box and regulatory region results in at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold increased mRNA expression of the one or more genes in the operon.

“ArgR” or “arginine repressor” is used to refer to a protein that is capable of suppressing arginine biosynthesis by regulating the transcription of arginine biosynthesis genes in the arginine regulon. When expression of the gene that encodes for the arginine repressor protein (“argR”) is increased in a wild-type bacterium, arginine biosynthesis is decreased. When expression of argR is decreased in a wild-type bacterium, or if argR is deleted or mutated to inactivate arginine repressor function, arginine biosynthesis is increased.

Bacteria that “lack any functional ArgR” and “ArgR deletion bacteria” are used to refer to bacteria in which each arginine repressor has significantly reduced or eliminated activity as compared to unmodified arginine repressor from bacteria of the same subtype under the same conditions. Reduced or eliminated arginine repressor activity can result in, for example, increased transcription of the arginine biosynthesis genes and/or increased concentrations of arginine and/or intermediate byproducts, e.g., citrulline. Bacteria in which arginine repressor activity is reduced or eliminated can be generated by modifying the bacterial argR gene or by modifying the transcription of the argR gene. For example, the chromosomal argR gene can be deleted, can be mutated, or the argR gene can be replaced with an argR gene that does not exhibit wild-type repressor activity.

“Operably linked” refers a nucleic acid sequence, e.g., a gene encoding feedback resistant ArgA, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region. In some embodiments, the genetically engineered bacteria of the invention comprise an oxygen level-dependent promoter induced by low-oxygen, microaerobic, or anaerobic conditions. In some embodiments, the genetically engineered bacteria comprise a promoter induced by a molecule or metabolite, for example, a tissue-specific molecule or metabolite or a molecule or metabolite indicative of liver damage. In some embodiments, the metabolites may be gut specific. In some embodiments, the metabolite may be associated with hepatic encephalopathy, e.g., bilirubin. Non-limiting examples of molecules or metabolites include, e.g., bilirubin, aspartate aminotransferase, alanine aminotransferase, blood coagulation factors II, VII, IX, and X, alkaline phosphatase, gamma glutamyl transferase, hepatitis antigens and antibodies, alpha fetoprotein, anti-mitochondrial, smooth muscle, and anti-nuclear antibodies, iron, transferrin, ferritin, copper, ceruloplasmin, ammonia, and manganese in their blood and intestines. Promoters that respond to one of these molecules or their metabolites may be used in the genetically engineered bacteria provided herein. In some embodiments, the genetically engineered bacteria comprise a promoter induced by inflammation or an inflammatory response, e.g., RNS or ROS promoter. In some embodiments, the genetically engineered bacteria comprise a promoter induced by a metabolite that may or may not be naturally present (e.g., can be exogenously added) in the gut, e.g., arabinose and tetracycline.

“Exogenous environmental condition(s)” refer to setting(s) or circumstance(s) under which the promoter described herein is induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, exogenous environmental conditions refer to the presence of molecules or metabolites that are specific to the mammalian gut in a healthy or disease state (e.g., HE). In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1
Examples of transcription factors and
responsive genes and regulatory regions

| Transcription | Exemplary responsive genes, promoters, |
|---|---|
| Factor | and/or regulatory regions: |
| FNR | nirB, ydfZ, pdhR, focA, ndH, hlyE, |
| | narK, narX, narG, yfiD, tdcD |
| ANR | arcDABC |
| DNR | norb, norC |

As used herein, a “gene cassette” or “operon” encoding a biosynthetic pathway refers to the two or more genes that are required to produce a gut barrier function enhancer molecule, e.g., butyrate, propionate. In addition to encoding a set of genes capable of producing said molecule, the gene cassette or operon may also comprise additional transcription and translation elements, e.g., a ribosome binding site.

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence, e.g., gene or gene cassette, may be present on a plasmid or bacterial chromosome. In some embodiments, the genetically engineered bacteria of the invention comprise a gene cassette that is operably linked to a directly or indirectly inducible promoter that is not associated with said gene cassette in nature, e.g., a FNR-responsive promoter operably linked to a butyrogenic gene cassette, or an arginine production cassette. In addition, multiple copies of the gene, gene cassette, or regulatory region may be present in the bacterium, wherein one or more copies may be mutated or otherwise altered as described herein. In some embodiments, the genetically engineered bacteria are engineered to comprise multiple copies of the same non-native nucleic acid sequence, e.g., gene, gene cassette, or regulatory region, in order to enhance copy number or to comprise multiple different components of a gene cassette performing multiple different functions.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

As used herein, genetically engineered bacteria that “overproduce” arginine or an intermediate byproduct, e.g., citrulline, refer to bacteria that comprise a mutant arginine regulon. For example, the engineered bacteria may comprise a feedback resistant form of ArgA, and when the arginine feedback resistant ArgA is expressed, are capable of producing more arginine and/or intermediate byproduct than unmodified bacteria of the same subtype under the same conditions. The genetically engineered bacteria may alternatively or further comprise a mutant arginine regulon comprising one or more nucleic acid mutations in at least one ARG box for each of the operons that encode the arginine biosynthesis enzymes. The genetically engineered bacteria may alternatively or further comprise a mutant or deleted arginine repressor. In some embodiments, the genetically engineered bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more arginine than unmodified bacteria of the same subtype under the same conditions. In some embodiments, the genetically engineered bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more citrulline or other intermediate byproduct than unmodified bacteria of the same subtype under the same conditions. In some embodiments, the mRNA transcript levels of one or more of the arginine biosynthesis genes in the genetically engineered bacteria are at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold higher than the mRNA transcript levels in unmodified bacteria of the same subtype under the same conditions. In certain embodiments, the unmodified bacteria will not have detectable levels of arginine, intermediate byproduct, and/or transcription of the gene(s) in such operons. However, protein and/or transcription levels of arginine and/or intermediate byproduct will be detectable in the corresponding genetically engineered bacterium having the mutant arginine regulon. Transcription levels may be detected by directly measuring mRNA levels of the genes. Methods of measuring arginine and/or intermediate byproduct levels, as well as the levels of transcript expressed from the arginine biosynthesis genes, are known in the art. Arginine and citrulline, for example, may be measured by mass spectrometry.

“Gut” refers to the organs, glands, tracts, and systems that are responsible for the transfer and digestion of food, absorption of nutrients, and excretion of waste. In humans, the gut comprises the gastrointestinal tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism”) to produce one or more therapeutic molecules. In certain aspects, the microorganism is engineered to import and/or catabolize certain toxic metabolites, substrates, or other compounds from its environment, e.g., the gut. In certain aspects, the microorganism is engineered to synthesize certain beneficial metabolites, molecules, or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are commensal bacteria. Examples of non-pathogenic bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

As used herein, “payload” refers to one or more polynucleotides and/or polypeptides of interest to be produced by a genetically engineered microorganism, such as a bacteria or a virus. In some embodiments, the payload is encoded by a gene or multiple genes or an operon. In some embodiments, the one or more genes and/or operon(s) comprising the payload are endogenous to the microorganism. In some embodiments, the one or more elements of the payload is derived from a different microorganism and/or organism. In some embodiments, the payload is a therapeutic payload. In some embodiments, the payload is encoded by genes for the biosynthesis of a molecule. In some embodiments, the payload is encoded by genes for the metabolism, catabolism, or degradation of a molecule. In some embodiments, the payload is encoded by genes for the importation of a molecule. In some embodiments, the payload is encoded by genes for the exportation of a molecule. In some embodiments, the payload is a regulatory molecule(s), e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads. Non-limiting examples of payload(s) include one or more of the following: (1) arginine racemase, (2) ArgAfbr, (3) mutated ArgR, (4) mutated ArgG. Other exemplary payloads include mutated sequence(s) that result in an auxotrophy, e.g., thyA auxotrophy, kill switch circuit, antibiotic resistance circuits, transporter sequence for importing biological molecules or substrates, secretion circuit.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., a feedback resistant argA gene, mutant arginine repressor, and/or other mutant arginine regulon that is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene encoding an arginine racemase, in which the plasmid or chromosome carrying the arginine racemase gene is stably maintained in the bacterium, such that arginine racemase can be expressed in the bacterium, and the bacterium is capable of survival and/or growth in vitro and/or in vivo.

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disorder, as well as those at risk of having, or who may ultimately acquire the disorder. The need for treatment is assessed, for example, by the presence of one or more risk factors associated with the development of a disorder, the presence or progression of a disorder, or likely receptiveness to treatment of a subject having the disorder. Primary hyperammonemia is caused by UCDs, which are autosomal recessive or X-linked inborn errors of metabolism for which there are no known cures. Hyperammonemia can also be secondary to other disruptions of the urea cycle, e.g., toxic metabolites, infections, and/or substrate deficiencies. Hyperammonemia can also contribute to other pathologies. For example, Huntington's disease is an autosomal dominant disorder for which there are no known cures. Urea cycle abnormalities characterized by hyperammonemia, high blood citrulline, and suppression of urea cycle enzymes may contribute to the pathology of Huntington's disease, an autosomal dominant disorder for which there are no known cures. Treating hyperammonemia may encompass reducing or eliminating excess ammonia and/or associated symptoms, and does not necessarily encompass the elimination of the underlying hyperammonemia-associated disorder.

As used herein a “pharmaceutical composition” refers to a preparation of genetically engineered bacteria of the invention with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., hyperammonemia. A therapeutically effective amount may, for example, be sufficient to treat, prevent, reduce the severity, delay the onset, and/or reduce the risk of occurrence of one or more symptoms of a disorder associated with elevated ammonia concentrations. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the protein of interest or therapeutic protein from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the protein(s) of interest or therapeutic protein(s) include a “secretion tag” of either RNA or peptide origin to direct the protein(s) of interest or therapeutic protein(s) to specific secretion systems. In some embodiments, the secretion system is able to remove this tag before secreting the protein(s) of interest or therapeutic protein(s) from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the protein(s) of interest or therapeutic protein(s) into the extracellular milieu.

As used herein, the term “transporter” is meant to refer to a mechanism, e.g., protein or proteins, for importing a molecule, e.g., amino acid, toxin, metabolite, substrate, etc. into the microorganism from the extracellular milieu.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

The genetically engineered bacteria disclosed herein are capable of reducing excess ammonia and converting ammonia and/or nitrogen into alternate byproducts. In some embodiments, the genetically engineered bacteria are naturally non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria are described in US Patent Publication US20160333326 and International Patent Publication WO2017139697, the contents of which is herein incorporated by reference in its entirety.

In some embodiments, the genetically engineered bacteria comprise circuitry in which one or more genes are under control of an inducible promoter. In some embodiments, the inducible promoter is a low-oxygen inducible promoter. In some embodiments, the promoter is inducible by inflammatory molecules, e.g., reactive nitrogen or reactive oxygen species (RNS or ROS). In some embodiments, the promoters are inducible by one or more nutritional and/or chemical inducer(s) and/or metabolite(s). Non-limiting examples of inducers include tetracycline, arabinose, IPTG, lactose, rhamnose, propionate.

In some embodiments, the genes are under control of a constitutive promoter. Suitable inducible promoters/promoter systems, and constitutive promoters are described for example in co-owned US Patent Publication US20160333326 and International Patent Publication WO2017139697, the contents of each of which is herein incorporated by reference in its entirety. In some embodiments, it is desirable to pre-induce activity of one or more ammonia catabolism circuitry components and/or other protein(s) of interest prior to administration. In such situations, the strains are pre-loaded with active payload or protein of interest. In such instances, the genetically engineered bacteria of the invention express one or more ammonia catabolism circuitry and/or other protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of different cell culture conditions, including anaerobic or low oxygen or oxygenated conditions, in the presence of inducers, nutrients, at defined temperatures, and the like. Methods for induction of ammonia strains are inter alia described in co-owned US Patent Publication US20160333326 and International Patent Publication WO2017139697, the contents of each of which is herein incorporated by reference in its entirety.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in one or more gene(s) required for cell survival and/or growth. Auxotrophic mutations are described in co-owned US Patent Publication US20160333326 and International Patent Publication WO2017139697, the contents of each of which is herein incorporated by reference in its entirety.

In some embodiments, the genetically engineered bacteria comprise multi-layered genetic regulatory circuits for expressing the constructs described herein. The genetic regulatory circuits are useful to screen for mutant bacteria that produce a component of an ammonia consuming circuitry or rescue an auxotroph. In certain embodiments, the invention provides methods for selecting genetically engineered bacteria that produce one or more genes of interest. Such regulatory circuitry is described in described in co-owned International Patent Publications WO2016/210378, US Patent Publication US20160333326 and International Patent Publication WO2017139697, the contents of each of which is herein incorporated by reference in its entirety.

In some embodiments, the genetically engineered bacteria also comprise a kill switch. The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death. Exemplary kill switches are described in co-owned International Patent Publications WO2016/210373, US Patent Publication US20160333326 and International Patent Publication WO2017139697, the contents of each of which is herein incorporated by reference in its entirety.

In some embodiments, the genetically engineered bacteria also comprise a plasmid that has been modified to create a host-plasmid mutual dependency. In certain embodiments, the mutually dependent host-plasmid platform. Examples of such platforms are described in Wright et al., 2015 GeneGuard: A Modular Plasmid System Designed for Biosafety; ACS Synth. Biol., 2015, 4 (3), pp 307-316, and in co-owned US Patent Publication US20160333326 and International Patent Publication WO2017139697, the contents of each of which is herein incorporated by reference in its entirety.

In some embodiments, any of the gene(s) or gene cassette(s) of the present disclosure may be integrated into the bacterial chromosome at one or more integration sites. One or more copies of the gene or gene cassette may be integrated into the bacterial chromosome. Having multiple copies of the gene or gene cassette integrated into the chromosome allows for greater production of the payload, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions. Exemplary integration sites, e.g. E coli Nissle integration sites are described in in co-owned US Patent Publication US20160333326 and International Patent Publication WO2017139697, the contents of each of which is herein incorporated by reference in its entirety.

In some embodiments, the genetically engineered bacteria further comprise a native secretion mechanism or non-native secretion mechanism that is capable of secreting a molecule from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane. Exemplary native and non-native secretion systems, secretion tags, diffusible outer membrane mutations and phenotypes, and methods and compositions useful for the secretion of active proteins are described in co-owned US Patent Publication US20160333326 and International Patent Publication WO2017139697, the contents of each of which is herein incorporated by reference in its entirety.

### Circuits

The disclosure provides genetically engineered bacteria that are capable of reducing excess ammonia and converting ammonia and/or nitrogen into alternate byproducts, including, but not limited to, L-arginine. In some embodiments, the engineered bacteria of the invention express a racemase which is capable of converting the byproducts, e.g., L-arginine to D-arginine. D-arginine and/or L-arginine can then be measured in the urine and/or feces of patients or of experimental animal models which are administered the engineered bacteria as an indication that the engineered bacteria are effectively converting ammonia to the byproduct, e.g., L-arginine, which can then be converted by the racemase to D-arginine.

As used herein, the term “racemase” refers to an enzyme which catalyzes the stereochemical inversion around the asymmetric carbon atom in a substrate having one center of asymmetry. In one embodiment, the term “amino acid racemase” refers to an enzyme which catalyzes the chemical reaction(s): L-amino acid [symbol] D-amino acid. Amino acid racemases are well known in the art and include, for example, EC 5.1.1.1 (alanine racemase), EC 5.1.1.2 (methionine racemase), EC 5.1.1.3 (glutamine racemase), EC 5.1.1.4 (proline racemase), EC 5.1.1.5 (lysine racemase), EC 5.1.1.6 (threonine racemase), EC 5.1.1.7 (diaminopimelate epimerase), EC 5.1.1.8 (4-hydroxyproline epimerase), EC 5.1.1.9 (arginine racemase), EC 5.1.1.10 (amino acid racemase), EC 5.1.1.11 (phenylalanine racemase), EC 5.1.1.12 (ornithine racemase), EC 5.1.1.13 (aspartate racemase), EC 5.1.1.14 (nocardicin-A epimerase), EC 5.1.1.15 (2-aminohexano-6-lactam racemase), EC 5.1.1.16 (protein-serine racemase), EC 5.1.1.17 (isopenicillin-N racemase), and EC 5.1.1.18 (serine racemase).

As also used herein, the term “arginine racemase’ refers to an enzyme which catalyzes the reaction L-arginine [symbol] D-arginine. Arginine racemases are well known in the art and include, for example, EC 5.1.1.9. See, for example, Matsui et al., Appl. Microbiol. Biotechnol. (2009) 83:1045-1054. In one embodiment, the arginine racemase is a pyridoxal-5′-phosphate-dependent amino acid racemase. In one embodiment, the arginine racemase is from Pseudomonas taetrolens.

In one embodiment, the arginine racemase is a d1-23 racemase. In another embodiment, the d1-23 racemase comprises a sequence disclosed in SEQ ID NO:5. In another embodiment, the d1-23 racemase lacks a signal peptide. In one embodiment, the d1-23 racemase further comprises the signal peptide sequence. In one embodiment, the d1-23 racemase is encoded by a sequence disclosed in SEQ ID NO:4. In one embodiment, the d1-23 racemase is encoded by a sequence which lacks a sequence encoding a signal peptide sequence. In one embodiment, the d1-23 racemase is encoded by a sequence which comprises a sequence encoding a signal peptide sequence. In one embodiment, the d1-23 racemase comprises a sequence or is encoded by a sequence having at least 60%, 65%, 70%, 75%, 80%, 85%, 90%, 91%, 92%, 93%, 94%, 95%, 96%, 97%, 98%, 99%, or 100% identity to a nucleic acid sequence or amino acid sequence disclosed herein.

In one embodiment, the arginine racemase is an ArR racemase. In another embodiment, the ArR racemase comprises a sequence disclosed in SEQ ID NO:12 or SEQ ID NO:14. In another embodiment, the ArR racemase lacks a signal peptide. In one embodiment, the ArR racemase further comprises the signal peptide sequence. An exemplary signal peptide sequence is disclosed herein as SEQ ID NO:13. In one embodiment, the ArR racemase is encoded by a sequence disclosed in SEQ ID NO:11 or SEQ ID NO:9. In one embodiment, the ArR racemase is encoded by a sequence which lacks a sequence encoding a signal peptide sequence. In one embodiment, the ArR racemase is encoded by a sequence which comprises a sequence encoding a signal peptide sequence. In one embodiment, a sequence encoding a signal peptide is disclosed herein as SEQ ID NO:10. In one embodiment, the ArR racemase comprises a sequence or is encoded by a sequence having at least 60%, 65%, 70%, 75%, 80%, 85%, 90%, 91%, 92%, 93%, 94%, 95%, 96%, 97%, 98%, 99%, or 100% identity to a nucleic acid sequence or amino acid sequence disclosed herein.

Typically, when a bacterial racemase is expressed, it comprises a signal peptide which allows it to localize into the periplasm of the bacteria. However, in some instances it may be useful to inhibit the localization of the racemase into the periplasm in order to increase the efficiency of the enzymatic reaction and/or increase access of the racemase to its substrates. Therefore, in one embodiment of the invention, the racemase comprises a signal peptide. In another embodiment, the racemase does not comprise a signal peptide. Signal peptide sequences are well known to one of ordinary skill in the art. Specific examples of signal peptide sequences include, but are not limited to, SEQ ID NO:13 and SEQ ID NO:10.

In some embodiments, the genetically engineered bacteria comprise one or more genes encoding a racemase. In some embodiments, the racemase is from Pseudomonas taetrolens. In some embodiments, the racemase expressed by the genetically engineered bacteria is localized to the periplasm. In some embodiments, the racemase expressed by the genetically engineered bacteria is localized to the cytoplasm. In some embodiments, the signal peptide which allows translocation to the periplasm, is deleted. In some embodiments, the first 23 amino acids of the translated racemase polypeptide are deleted. In some embodiments, the first 69 nucleotides of the racemase gene sequence are deleted.

In some embodiments, the genetically engineered bacteria express a racemase from a plasmid and/or chromosome. In some embodiments, the gene encoding the racemase is expressed under the control of a constitutive promoter. Suitable constitutive promoter systems are described in co-owned WO2017139697 and US20160333326, the contents of which is herein incorporated by reference in its entirety. In some embodiments, the gene encoding the racemase is under the control of an inducible promoter. In some embodiments, the gene encoding the racemase is under the control of a promoter induced by reactive oxygen species. In some embodiments, gene encoding the racemase is under the control of a promoter induced by reactive nitrogen species. In some embodiments, the gene encoding the racemase is under control of a low oxygen inducible promoter. In some embodiments, the gene encoding the racemase is under the control of an FNR, ANR, or DNR inducible promoter. In one embodiment, the gene encoding the racemase is under control of an FNR inducible promoter. In one specific embodiment, the FNR promoter is FNRS. In some embodiments, the gene encoding the racemase is inducible by a chemical or nutritional inducer, e.g., tetracycline, arabinose, IPTG, rhamnose and others. Suitable inducible promoter systems are described in co-owned WO2017139697 and US20160333326, the contents of which is herein incorporated by reference in its entirety. In some embodiments, the gene encoding the racemase is present on a high copy plasmid. In some embodiments, the gene encoding the racemase is present on a low copy plasmid. In some embodiments, the gene encoding the racemase is integrated into the chromosome. Suitable integration sites are described in co-owned WO2017139697 and US20160333326, the contents of which is herein incorporated by reference in its entirety.

In some embodiments, the genetically engineered bacteria comprising a gene encoding arginine racemase further comprise an arginine feedback resistant N-acetylglutamate synthetase (ArgA^{fbr}). Feedback resistant forms of ArgA are described in in co-owned WO2017139697, the contents of which is herein incorporated by reference in its entirety. In some embodiments, the genetically engineered bacteria comprise feedback-resistant carbamoyl-phosphate synthetase (ArgAfbr). In some embodiments, the genetically engineered bacteria express ArgA^{fbr} from a plasmid and/or chromosome. In some embodiments, the ArgA^{fbr} gene is expressed under the control of a constitutive promoter. Suitable constitutive promoter systems are described in co-owned WO2017139697, the contents of which is herein incorporated by reference in its entirety. In some embodiments, ArgA^{fbr} is under the control of an inducible promoter. In some embodiments, ArgA^{fbr} is under the control of a promoter induced by reactive oxygen species. In some embodiments, ArgA^{fbr} is under the control of a promoter induced by reactive nitrogen species. In some embodiments, ArgA^{fbr} is under control of a low oxygen inducible promoter. In some embodiments, ArgA^{fbr} is under the control of an FNR, ANR, or DNR inducible promoter. In one embodiment, ArgA^{fbr} is under control of an FNR inducible promoter. In one specific embodiment, the FNR promoter is FNRS. In some embodiments, ArgA^{fbr} is inducible by a chemical or nutritional inducer, e.g., tetracycline, arabinose, IPTG, rhamnose and others. Suitable inducible promoter systems are described in co-owned WO2017139697, the contents of which is herein incorporated by reference in its entirety. In some embodiments, ArgA^{fbr} is present on a high copy plasmid. In some embodiments, ArgA^{fbr} is present on a low copy plasmid. In some embodiments, is integrated into the chromosome. Suitable integration sites are described in co-owned WO2017139697, the contents of which is herein incorporated by reference in its entirety.

In some embodiments, the genetically engineered bacteria comprising a gene encoding arginine racemase further comprise a deletion in ArgR. In some embodiments, the genetically engineered bacteria comprising a gene encoding arginine racemase and ArgA^{fbr} further comprise a deletion or mutation in ArgR, rendering ArgR non-functional, such that it can no longer repress ArgA^{fbr}.

In some embodiments, the strain comprising a gene encoding arginine racemase further includes an auxotrophy. In some embodiments, the strain comprising a gene encoding arginine racemase further includes an auxotrophy in thyA. In some embodiments, the strain comprising a gene encoding arginine racemase and ArgA^{fbr} further includes an auxotrophy in thyA. In some embodiments, the strain comprising a gene encoding arginine racemase, ArgA^{fbr}, and ΔArgR further includes an auxotrophy in thyA.

### Pharmaceutical Compositions

Pharmaceutical compositions comprising the genetically engineered microorganisms of the invention may be used to treat, manage, ameliorate, and/or prevent a disorder associated with hyperammonemia or symptom(s) associated with diseases or disorders associated with hyperammonemia. Pharmaceutical compositions of the invention comprising one or more genetically engineered bacteria, and/or one or more genetically engineered yeast or virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise one or more of the genetic modifications described herein, e.g., selected from expression of at least one ammonium consuming circuit component, auxotrophy, kill-switch, exporter knock-out, etc. In alternate embodiments, the pharmaceutical composition comprises two or more species, strains, and/or subtypes of bacteria that are each engineered to comprise the genetic modifications described herein, e.g., selected from expression of at least one ammonia consuming circuit, auxotrophy, kill-switch, exporter knock-out, etc.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal. Suitable pharmaceutical compositions and methods of administration are for example described in in co-owned US Patent Publication US20160333326 and International Patent Publication WO2017139697, the contents of each of which is herein incorporated by reference in its entirety. Methods of Screening, including Generation of Bacterial Strains with Enhance Ability to consume ammonia, are for example described in in co-owned US Patent Publication US20160333326 and International Patent Publication WO2017139697, the contents of each of which is herein incorporated by reference in its entirety.

Strains comprising Feedback Resistant N-acetylglutamate Synthetase, inducible constructs thereof, and sequences are described in US Patent Publication US20160333326 and International Patent Publication WO2017139697, the contents of which is herein incorporated by reference in its entirety. Mutations and or deletions in ArgR are described in in US Patent Publication US20160333326 and International Patent Publication WO2017139697, the contents of which is herein incorporated by reference in its entirety. Such constructs mutations, and deletions may be used in strains of the current disclosure.

### Methods of Treatment

The disclosure provides genetically engineered bacteria that are capable of reducing excess ammonia and converting ammonia and/or nitrogen into alternate byproducts. In some embodiments, the engineered bacteria of the disclosure express a racemase which is capable of converting a byproduct, e.g., L-arginine to D-arginine. D-arginine and/or L-arginine can then be measured in the urine and/or feces of patients or of an experimental animal model which are administered the engineered bacteria as an indication that the engineered bacteria are effectively converting ammonia through to D-arginine. The addition of a racemase enzyme to the genetically engineered bacteria is useful for production of a detectable biomarker that can be used to easily assess the activity of the strain.

Methods for determining the presence of arginine, e.g., D-arginine and/or L-arginine, in urine and feces are known in the art and include, for example, ion exchange chromatography, high-pressure liquid chromatography, and tandem mass spectrometry. For example, in one embodiment, the quantification of plasma and urinary amino acids (arginine, methionine, ornithine, and glycine) may be carried out with a Biochrom 30 ionic chromatograph (Gomensoro, Madrid). The instrument has a specific program to separate the amino acids using Biochrom Ultropak 4 and Ultropak 8 columns. The mobile phases are commercialized as a kit (Biochrom Reference 80-2098-05). After post column derivatization with ninhydrin, the absorbance is monitored at 440 and 570 nm.

Another aspect of the invention provides methods of treating a disease or disorder associated with hyperammonemia. In some embodiments, the invention provides methods for reducing, ameliorating, or eliminating one or more symptom(s) associated with these diseases or disorders. In some embodiments, the disorder is a urea cycle disorder such as argininosuccinic aciduria, arginase deficiency, carbamoylphosphate synthetase deficiency, citrullinemia, N-acetylglutamate synthetase deficiency, and ornithine transcarbamylase deficiency. In alternate embodiments, the disorder is a liver disorder such as hepatic encephalopathy, acute liver failure, or chronic liver failure; organic acid disorders; isovaleric aciduria; 3-methylcrotonylglycinuria; methylmalonic acidemia; propionic aciduria; fatty acid oxidation defects; carnitine cycle defects; carnitine deficiency; β-oxidation deficiency; lysinuric protein intolerance; pyrroline-5-carboxylate synthetase deficiency; pyruvate carboxylase deficiency; ornithine aminotransferase deficiency; carbonic anhydrase deficiency; hyperinsulinism-hyperammonemia syndrome; mitochondrial disorders; valproate therapy; asparaginase therapy; total parenteral nutrition; cystoscopy with glycine-containing solutions; post-lung/bone marrow transplantation; portosystemic shunting; urinary tract infections; ureter dilation; multiple myeloma; chemotherapy; infection; neurogenic bladder; or intestinal bacterial overgrowth. In some embodiments, the symptom(s) associated thereof include, but are not limited to, seizures, ataxia, stroke-like lesions, coma, psychosis, vision loss, acute encephalopathy, cerebral edema, as well as vomiting, respiratory alkalosis, and hypothermia.

The method may comprise preparing a pharmaceutical composition with at least one genetically engineered species, strain, or subtype of bacteria described herein, and administering the pharmaceutical composition to a subject in a therapeutically effective amount. In some embodiments, the genetically engineered bacteria of the invention are administered orally, e.g., in a liquid suspension. In some embodiments, the genetically engineered bacteria of the invention are lyophilized in a gel cap and administered orally. In some embodiments, the genetically engineered bacteria of the invention are administered via a feeding tube or gastric shunt. In some embodiments, the genetically engineered bacteria of the invention are administered rectally, e.g., by enema. In some embodiments, the genetically engineered bacteria of the invention are administered topically, intraintestinally, intrajejunally, intraduodenally, intraileally, and/or intracolically.

In certain embodiments, administering the pharmaceutical composition to the subject reduces ammonia concentrations in a subject. In some embodiments, the methods of the present disclosure may reduce the ammonia concentration in a subject by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing the ammonia concentration in a subject before and after administration of the pharmaceutical composition. In some embodiments, the method of treating or ameliorating hyperammonemia allows one or more symptoms of the condition or disorder to improve by at least about 10%, 20%, 30%, 40%, 50%, 60%, 70%, 80%, 90%, 95%, or more.

Before, during, and after the administration of the pharmaceutical composition, ammonia concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions of the invention to reduce ammonia concentrations in a subject to undetectable levels, or to less than about 1%, 2%, 5%, 10%, 20%, 25%, 30%, 40%, 50%, 60%, 70%, 75%, or 80% of the subject's ammonia concentrations prior to treatment.

In some embodiments, the engineered bacteria express a racemase which is capable of converting, e.g., L-arginine to D-arginine. D-arginine and/or L-arginine levels in a sample from the subject can then be measured before, during and/or after administration of the pharmaceutical composition as an indication that the engineered bacteria are effectively converting ammonia to L-arginine, and then to D-arginine.

In some embodiments, the methods may include administration of the compositions of the invention resulting in the production of D-arginine concentrations of at least about 1.2 to 1.4-fold, at least about 1.4 to 1.6-fold, at least about 1.6 to 1.8-fold, at least about 1.8 to 2-fold, or at least about 2 to 3-fold, at least about 3 to 4-fold, at least about 4 to 5-fold, at least about 5 to 6-fold, at least about 6 to 7-fold, or at least about 7 to 8-fold more D-arginine than the subject's D-arginine concentrations prior to treatment.

In any of these embodiments, the genetically engineered bacteria comprising gene sequences encoding a racemase may produce at least about 0% to 2%, at least about 2% to 4%, at least about 4% to 6%, at least about 6% to 8%, at least about 8% to 10%, at least about 10% to 12%, at least about 12% to 14%, at least about 14% to 16%, at least about 16% to 18%, at least about 18% to 20%, at least about 20% to 25%, at least about 25% to 30%, at least about 30% to 35%, at least about 35% to 40%, at least about 40% to 45%, at least about 45% to 50%, at least about 50% to 55%, at least about 55% to 60%, at least about 60% to 65%, at least about 65% to 70% to 80%, at least about 80% to 90%, or at least about 90% to 100% more D-arginine than bacteria that do not comprise gene sequences encoding a racemase of the same bacterial subtype under the same conditions. In yet another embodiment, the genetically engineered bacteria comprising gene sequences encoding a racemase may produce at least about 1.0 to 1.2-fold, at least about 1.2 to 1.4-fold, at least about 1.4 to 1.6-fold, at least about 1.6 to 1.8-fold, at least about 1.8 to 2-fold, or at least about two-fold more D-arginine than bacteria that do not comprise gene sequences encoding a racemase of the same bacterial subtype under the same conditions. In yet another embodiment, the genetically engineered bacteria comprising gene sequences encoding a racemase produce at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, at least about 40 to 50-fold, at least about 50 to 100-fold, 100 to 500 hundred-fold, or at least about 500 to 1000-fold more D-arginine than bacteria that do not comprise gene sequences encoding a racemase of the same bacterial subtype under the same conditions. In some embodiments, the conditions are in vitro conditions, e.g., during bacterial growth in culture. In some embodiments, the conditions are in vivo conditions, e.g., in the gut after administration of the bacteria to a subject (e.g., human, mouse or non-human primate).

In any of these embodiments, at least about 0% to 2%, at least about 2% to 4%, at least about 4% to 6%, at least about 6% to 8%, at least about 8% to 10%, at least about 10% to 12%, at least about 12% to 14%, at least about 14% to 16%, at least about 16% to 18%, at least about 18% to 20%, at least about 20% to 25%, at least about 25% to 30%, at least about 30% to 35%, at least about 35% to 40%, at least about 40% to 45%, at least about 45% to 50%, at least about 50% to 55%, at least about 55% to 60%, at least about 60% to 65%, at least about 65% to 70%, at least about 70% to 80%, at least about 80% to 90%, or at least about 90% to 100% more D-arginine is detected in the plasma of a subject (e.g., human, mouse or non-human primate) upon administration of the genetically engineered bacteria comprising gene sequences encoding a racemase than upon administration of bacteria that do not comprise gene sequences encoding a racemase of the same bacterial subtype under the same conditions. In yet another embodiment, at least about 1.0-1.2-fold, at least about 1.2-1.4-fold, at least about 1.4-1.6-fold, at least about 1.6-1.8-fold, at least about 1.8-2-fold, or at least about two-fold or more D-arginine is detected in the plasma upon administration of the genetically engineered bacteria comprising gene sequences encoding a racemase than upon administration of bacteria that do not comprise gene sequences encoding a racemase of the same bacterial subtype under the same conditions. In yet another embodiment, at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold more D-arginine is detected in the plasma upon administration of the genetically engineered bacteria comprising gene sequences encoding a racemase than upon administration of bacteria that do not comprise gene sequences encoding a racemase of the same bacterial subtype under the same conditions. In one embodiment, about 2-fold more plasma D-Arginine is detected in the plasma upon administration of the genetically engineered bacteria comprising gene sequences encoding a racemase than upon administration of bacteria that do not comprise gene sequences encoding a racemase of the same bacterial subtype under the same conditions, e.g., after 1, 2, 3, 4, 5, and/or 6 hours.

In some embodiments, the area under the curve is calculated after plasma D-arginine is measured over a timeframe. In some embodiments, the AUC is at least about 1 to 2-fold, at least about 2 to 3-fold, at least about 3 to 4-fold, or at least about 4 to 5-fold higher upon administration of the genetically engineered bacteria comprising gene sequences encoding a racemase than upon administration of bacteria that do not comprise gene sequences encoding a racemase of the same bacterial subtype under the same conditions. In one embodiment, the time frame is 6 hours. In one embodiment, the AUC is at least about 2 to 3 fold higher upon administration of the genetically engineered bacteria comprising gene sequences encoding a racemase than upon administration of bacteria that do not comprise gene sequences encoding a racemase of the same bacterial subtype under the same conditions.

In some embodiments, the plasma D-arginine levels are measured after about 10, about 20, about 30, about 40, about 50 and/or about 60 minutes. In some embodiments, the plasma D-arginine levels are measured after about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, and/or about 24 hours. In some embodiments, the plasma D-arginine levels are measured between about 1 and 2, about 2 and 3, about 3 and 4, about 4 and 5, about 5 and 6, and/or about 6 and 7 hours. In some embodiments, the plasma D-arginine levels are measured after about 1, about 2, about 3, about 4, about 5, about 6, and/or about 7 days, or after about 1, about 2, about 3, and/or about 4 weeks, or after about 1, about 2, about 3, about 4, about 5, about 6, about 7, about 8, about 9, about 10, about 11, about 12 months after administration. In some embodiments, the plasma D-arginine levels are measured after one or more years after administration. In one embodiment, the plasma D-arginine levels are measured after about 1, 2, 3, 4, 5, and 6 hours.

In any of these embodiments, at least about 0% to 2%, at least about 2% to 4%, at least about 4% to 6%, at least about 6% to 8%, at least about 8% to 10%, at least about 10% to 12%, at least about 12% to 14%, at least about 14% to 16%, at least about 16% to 18%, at least about 18% to 20%, at least about 20% to 25%, at least about 25% to 30%, at least about 30% to 35%, at least about 35% to 40%, at least about 40% to 45%, at least about 45% to 50%, at least about 50% to 55%, at least about 55% to 60%, at least about 60% to 65%, at least about 65% to 70%, at least about 70% to 80%, at least about 80% to 90%, or at least about 90% to 100% more D-arginine is detected in the urine of a subject (e.g., human, mouse or non-human primate) upon administration of the genetically engineered bacteria comprising gene sequences encoding a racemase than upon administration of bacteria that do not comprise gene sequences encoding a racemase of the same bacterial subtype under the same conditions. In yet another embodiment, at least about 1.0-1.2-fold, at least about 1.2-1.4-fold, at least about 1.4-1.6-fold, at least about 1.6-1.8-fold, at least about 1.8-2-fold, or at least about two-fold or more D-arginine is detected in the urine upon administration of the genetically engineered bacteria comprising gene sequences encoding a racemase than upon administration of bacteria that do not comprise gene sequences encoding a racemase of the same bacterial subtype under the same conditions. In yet another embodiment, at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold more D-arginine is detected in the urine upon administration of the genetically engineered bacteria comprising gene sequences encoding a racemase than upon administration of bacteria that do not comprise gene sequences encoding a racemase of the same bacterial subtype under the same conditions. In one embodiment, about 6 to 7-fold more urine D-Arginine is detected in the urine upon administration of the genetically engineered bacteria comprising gene sequences encoding a racemase than upon administration of bacteria that do not comprise gene sequences encoding a racemase of the same bacterial subtype under the same conditions, e.g., after 6 hours.

In some embodiments, the area under the curve is calculated after urine D-arginine is measured over a timeframe. In some embodiments, the AUC is at least about 1 to 2-fold, at least about 2 to 3-fold, at least about 3 to 4-fold, or at least about 4 to 5-fold higher upon administration of the genetically engineered bacteria comprising gene sequences encoding a racemase than upon administration of bacteria that do not comprise gene sequences encoding a racemase of the same bacterial subtype under the same conditions. In one embodiment, the time frame is 6 hours. In one embodiment, the AUC is at least about 2 to 3-fold higher upon administration of the genetically engineered bacteria comprising gene sequences encoding a racemase than upon administration of bacteria that do not comprise gene sequences encoding a racemase of the same bacterial subtype under the same conditions.

In some embodiments, the urine D-arginine levels are measured after about 10, about 20, about 30, about 40, about 50 and/or about 60 minutes. In some embodiments, the urine D-arginine levels are measured after about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, and/or about 24 hours. In some embodiments, the urine D-arginine levels are measured between about 1 and 2, about 2 and 3, about 3 and 4, about 4 and 5, about 5 and 6, and/or about 6 and 7 hours. In some embodiments, the urine D-arginine levels are measured after about 1, about 2, about 3, about 4, about 5, about 6, and/or about 7 days, or after about 1, about 2, about 3, and/or about 4 weeks, or after about 1, about 2, about 3, about 4, about 5, about 6, about 7, about 8, about 9, about 10, about 11, about 12 months after administration. In some embodiments, the urine D-arginine levels are measured after one or more years after administration. In one embodiment, the urine D-arginine levels are measured after about 1, 2, 3, 4, 5, and 6 hours.

In one embodiment, the level of D-arginine in the urine and/or feces of the subject increases from a concentration of about 0.001 mM before administration to about 0.6 mM about 3 hours after administration. In another embodiment, the level of D-arginine in the urine and/or feces of the subject increases from a concentration of about 0.001 mM before administration to about 0.3 mM about 2 hours after administration. In another embodiment, the level of D-arginine in the urine and/or feces of the subject increases from a concentration of about 0.001 mM before administration to about 0.1 mM about 1 hour after administration.

In one embodiment, the level of L-arginine in the urine and/or feces of a subject increases from a concentration of about 0.001 mM before administration to about 0.65 mM about 3 hours after administration. In another embodiment, the level of L-arginine in the urine and/or feces of a subject increases from a concentration of about 0.001 mM before administration to about 0.39 mM about 3 hours after administration. In another embodiment, the level of L-arginine in the urine and/or feces of a subject increases from a concentration of about 0.001 mM before administration to about 0.19 mM about 3 hours after administration.

In some embodiments, the genetically engineered bacteria comprising gene sequences encoding a racemase are administered once. In some embodiments, the genetically engineered bacteria comprising gene sequences encoding a racemase are administered more than once (e.g., more than once daily, more than once weekly, more than once monthly). In some embodiments, the genetically engineered bacteria comprising gene sequences encoding a racemase are administered more than once (e.g., twice daily or more, or 2, 3, 4, 5, 6, 7, 8, 9, 10, 11, 12, 13, 14 times or more weekly. In some embodiments, the genetically engineered bacteria comprising gene sequences encoding a racemase are administered once, twice or more daily for one or more months. In some embodiments, the genetically engineered bacteria comprising gene sequences encoding a racemase are administered once, twice or more daily for one or more years.

In some embodiments, the genetically engineered bacteria comprise gene sequences encoding racemase and feedback resistance argA (ArgAfbr) and comprise a deletion in argR. In any of these embodiments, the genetically engineered bacteria comprising gene sequences encoding racemase and feedback resistance argA (ArgAfbr) and comprising a deletion in argR may produce at least about 0% to 2%, at least about 2% to 4%, at least about 4% to 6%, at least about 6% to 8%, at least about 8% to 10%, at least about 10% to 12%, at least about 12% to 14%, at least about 14% to 16%, at least about 16% to 18%, at least about 18% to 20%, at least about 20% to 25%, at least about 25% to 30%, at least about 30% to 35%, at least about 35% to 40%, at least about 40% to 45%, at least about 45% to 50%, at least about 50% to 55%, at least about 55% to 60%, at least about 60% to 65%, at least about 65% to 70% to 80%, at least about 80% to 90%, or at least about 90% to 100% more D-arginine than bacteria engineered to encode sequences for the expression of racemase alone of the same bacterial subtype under the same conditions. In yet another embodiment, the genetically engineered bacteria comprising gene sequences encoding racemase and feedback resistance argA (ArgAfbr) and comprising a deletion in argR produce at least about 1.0 to 1.2-fold, at least about 1.2 to 1.4-fold, at least about 1.4 to 1.6-fold, at least about 1.6 to 1.8-fold, at least about 1.8 to 2-fold, or at least about two-fold more D-arginine than bacteria engineered to encode sequences for the expression of racemase alone of the same bacterial subtype under the same conditions. In yet another embodiment, the genetically engineered bacteria comprising gene sequences encoding racemase and feedback resistance argA (ArgAfbr) and comprising a deletion in argR produce at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, at least about 40 to 50-fold, at least about 50 to 100-fold, 100 to 500 hundred-fold, or at least about 500 to 1000-fold more D-arginine than bacteria engineered to encode sequences for the expression of racemase alone of the same bacterial subtype under the same conditions. In some embodiments, the conditions are in vitro conditions, e.g., during bacterial growth in culture. In some embodiments, the conditions are in vivo conditions, e.g., in the gut after administration of the bacteria to a subject (e.g., human, mouse or non-human primate).

In any of these embodiments, at least about 0% to 2%, at least about 2% to 4%, at least about 4% to 6%, at least about 6% to 8%, at least about 8% to 10%, at least about 10% to 12%, at least about 12% to 14%, at least about 14% to 16%, at least about 16% to 18%, at least about 18% to 20%, at least about 20% to 25%, at least about 25% to 30%, at least about 30% to 35%, at least about 35% to 40%, at least about 40% to 45%, at least about 45% to 50%, at least about 50% to 55%, at least about 55% to 60%, at least about 60% to 65%, at least about 65% to 70%, at least about 70% to 80%, at least about 80% to 90%, or at least about 90% to 100% more D-arginine is detected in the plasma of a subject (e.g., human, mouse or non-human primate) upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In yet another embodiment, at least about 1.0-1.2-fold, at least about 1.2-1.4-fold, at least about 1.4-1.6-fold, at least about 1.6-1.8-fold, at least about 1.8-2-fold, or at least about two-fold or more D-arginine is detected in the plasma upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In yet another embodiment, at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold more D-arginine is detected in the plasma upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In one embodiment, about 2-fold more plasma D-Arginine is detected in the plasma upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions, e.g., after 1, 2, 3, 4, 5, and/or 6 hours.

In some embodiments, the area under the curve is calculated after plasma D-arginine is measured over a timeframe. In some embodiments, the AUC is at least about 1 to 2-fold, at least about 2 to 3-fold, at least about 3 to 4-fold, or at least about 4 to 5-fold higher upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In one embodiment, the time frame is 6 hours. In one embodiment, the AUC is at least about 2 to 3-fold higher upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In one embodiment, the area under the curve for detected plasma D-arg is about 2 to 2.5-fold or 2.5 to 3-fold greater in the plasma upon administration of the genetically engineered bacteria comprising gene sequences encoding a racemase than upon administration of bacteria that do not comprise gene sequences encoding a racemase of the same bacterial subtype under the same conditions, e.g., after 1, 2, 3, 4, 5, and/or 6 hours.

In some embodiments, the plasma D-arginine levels are measured after about 10, about 20, about 30, about 40, about 50 and/or about 60 minutes. In some embodiments, the plasma D-arginine levels are measured after about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, and/or about 24 hours. In some embodiments, the plasma D-arginine levels are measured between about 1 and 2, about 2 and 3, about 3 and 4, about 4 and 5, about 5 and 6, and/or about 6 and 7 hours. In some embodiments, the plasma D-arginine levels are measured after about 1, about 2, about 3, about 4, about 5, about 6, and/or about 7 days, or after about 1, about 2, about 3, and/or about 4 weeks, or after about 1, about 2, about 3, about 4, about 5, about 6, about 7, about 8, about 9, about 10, about 11, about 12 months after administration. In some embodiments, the plasma D-arginine levels are measured after one or more years after administration. In one embodiment, the plasma D-arginine levels are measured after about 1, 2, 3, 4, 5, and 6 hours.

In any of these embodiments, at least about 0% to 2%, at least about 2% to 4%, at least about 4% to 6%, at least about 6% to 8%, at least about 8% to 10%, at least about 10% to 12%, at least about 12% to 14%, at least about 14% to 16%, at least about 16% to 18%, at least about 18% to 20%, at least about 20% to 25%, at least about 25% to 30%, at least about 30% to 35%, at least about 35% to 40%, at least about 40% to 45%, at least about 45% to 50%, at least about 50% to 55%, at least about 55% to 60%, at least about 60% to 65%, at least about 65% to 70%, at least about 70% to 80%, at least about 80% to 90%, or at least about 90% to 100% more D-arginine is detected in the urine of a subject (e.g., human, mouse or non-human primate) upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In yet another embodiment, at least about 1.0-1.2-fold, at least about 1.2-1.4-fold, at least about 1.4-1.6-fold, at least about 1.6-1.8-fold, at least about 1.8-2-fold, or at least about two-fold or more D-arginine is detected in the urine upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In yet another embodiment, at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold more D-arginine is detected in the urine upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In one embodiment, about 6 to 7-fold more urine D-Arginine is detected in the urine upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions, e.g., after 6 hours.

In some embodiments, the area under the curve is calculated after urine D-arginine is measured over a timeframe. In some embodiments, the AUC is at least about 1 to 2-fold, at least about 2 to 3-fold, at least about 3 to 4-fold, or at least about 4 to 5-fold higher upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions. In one embodiment, the time frame is 6 hours. In one embodiment, the AUC is at least about 2 to 3-fold higher upon administration of the genetically engineered bacteria comprising gene sequences encoding racemase and feedback resistance argA (ArgAfbr) and comprising a deletion in argR than upon administration of bacteria engineered to encode sequences for the expression of racemase alone of the same bacterial subtype under the same conditions.

In some embodiments, the urine D-arginine levels are measured after about 10, about 20, about 30, about 40, about 50 and/or about 60 minutes. In some embodiments, the urine D-arginine levels are measured after about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, and/or about 24 hours. In some embodiments, the urine D-arginine levels are measured between about 1 and 2, about 2 and 3, about 3 and 4, about 4 and 5, about 5 and 6, and/or about 6 and 7 hours. In some embodiments, the urine D-arginine levels are measured after about 1, about 2, about 3, about 4, about 5, about 6, and/or about 7 days, or after about 1, about 2, about 3, and/or about 4 weeks, or after about 1, about 2, about 3, about 4, about 5, about 6, about 7, about 8, about 9, about 10, about 11, about 12 months after administration. In some embodiments, the urine D-arginine levels are measured after one or more years after administration. In one embodiment, the urine D-arginine levels are measured after about 1, 2, 3, 4, 5, and 6 hours.

In some embodiments, the genetically engineered bacteria comprising gene sequences encoding racemase and feedback resistance argA (ArgAfbr) and comprising a deletion in argR are administered once. In some embodiments, the genetically engineered bacteria comprising gene sequences encoding racemase and feedback resistance argA (ArgAfbr) and comprising a deletion in argR are administered more than once (e.g., more than once daily, more than once weekly, more than once monthly). In some embodiments, the genetically engineered bacteria comprising gene sequences encoding racemase and feedback resistance argA (ArgAfbr) and comprising a deletion in argR are administered more than once (e.g., twice daily or more, or 2, 3, 4, 5, 6, 7, 8, 9, 10, 11, 12, 13, 14 times or more weekly. In some embodiments, the genetically engineered bacteria comprising gene sequences encoding racemase and feedback resistance argA (ArgAfbr) and comprising a deletion in argR are administered once, twice or more daily for one or more months. In some embodiments, the genetically engineered bacteria comprising gene sequences encoding racemase and feedback resistance argA (ArgAfbr) and comprising a deletion in argR are administered once, twice or more daily for one or more years.

In any of these embodiments, the amount of D-arginine produced by the genetically engineered bacteria expressing racemase and ArgAfbr and comprising a deletion in ArgR over the bacteria engineered to express racemase alone can be used as an indicator of strain activity, e.g., as an indicator of activity of a separate strain comprising gene sequences encoding ArgAfbr and comprising a deletion in ArgR of the same bacterial subtype under the same conditions.

Accordingly, methods are provided herein which allow the activity of a genetically engineered bacterium comprising a circuitry for the production of certain metabolite(s) to be measured. Such methods include the addition of an enzyme into the strain itself, such as a racemase, which can convert the metabolite into a corresponding non-naturally occurring metabolite, whose levels are subsequently measured. Alternatively, a second surrogate strain of the same subtype can be constructed, comprising the circuitry of interest for the production of certain metabolite(s) and additionally comprising the circuitry for expression of the enzyme that converts the metabolite into a non-naturally occurring metabolite. In both cases, the accumulation of the non-naturally occurring metabolite is measured under set conditions and is used as an indicator of the activity of the metabolite producing circuit of interest.

In a non-limiting example, a first engineered bacterium, whose activity is measured by the methods, comprises circuitry for the production an amino acid. Accordingly, a second strain of the same subtype which comprises said circuitry and additionally an enzyme such as a racemase, which converts the amino acid from the L to the D-form, can be used to assess the activity the metabolite producing circuitry of said first strain under the same conditions by measuring the accumulation of the non-naturally occurring D-form. Levels of the D-form of the amino acid may be used as an indicator of activity of any circuitry of interest in the strain. In an alternate embodiment, the enzyme for production of the non-natural substrate is added directly to the first strain.

In one embodiment, a method described herein allows the measurement of activity an arginine producing strain. The strain may comprise ArgAfbr, e.g., under control of a low oxygen promoter, and a deletion in argR. A non-limiting example of a strain of the same subtype that can be used as an indicator for activity under same conditions is a strain comprising ArgAfbr, e.g., under control of a low oxygen promoter, and a deletion in argR and a racemase for the conversion of L-Arg to D-arg. One non-limiting examples of such a s racemase is Pseudomonas taetrolens racemase.

In certain embodiments, the genetically engineered bacteria comprising circuitry for the expression of arginine and optionally the mutant arginine regulon is E. coli Nissle. The genetically engineered bacteria may be destroyed, e.g., by defense factors in the gut or blood serum (Sonnenborn et al., 2009), or by activation of a kill switch, several hours or days after administration. Thus, the pharmaceutical composition comprising the mutant arginine regulon may be re-administered at a therapeutically effective dose and frequency. In alternate embodiments, the genetically engineered bacteria are not destroyed within hours or days after administration and may propagate and colonize the gut.

The pharmaceutical composition may be administered alone or in combination with one or more additional therapeutic agents, including but not limited to, sodium phenylbutyrate, sodium benzoate, and glycerol phenylbutyrate. An important consideration in the selection of the one or more additional therapeutic agents is that the agent(s) should be compatible with the genetically engineered bacteria of the invention, e.g., the agent(s) must not kill the bacteria.

In one embodiment, the genetically engineered bacteria are administered for prevention, treatment or management of HE. In some embodiments, the genetically engineered bacteria are administered in combination with another therapeutic approach to prevent HE reoccurrence. In one embodiment, the genetically engineered bacteria are administered in combination with branched-chain amino acid supplementation. In one embodiment, the genetically engineered bacteria are administered in combination with acetyl-1-carnitine and/or sodium benzoate and/or zinc and/or acarbose and/or ornithine aspartate. In one embodiment, the genetically engineered bacteria are administered in combination with non-absorbable disaccharides, which are commonly applied to both treat and prevent HE in patients. In one embodiment, the genetically engineered bacteria are administered in combination with lactulose and/or lactitol.

In one embodiment, the genetically engineered bacteria are administered in combination with one or more antibiotics, for example for the treatment of HE. Examples of such antibiotics include, but are not limited to, non-absorbable antibiotics, such as aminoglicosides, e.g., neomycin and/or paramomycin. In one embodiment, the antibiotic is rifamycin. In one embodiment, the antibiotic is a rifamycin derivative, e.g., a synthetic derivative, including but not limited to, rifaximin.

Rifaximin has been shown to significantly reduce the risk of an episode of hepatic encephalopathy, as compared with placebo, over a 6-month period (Bass et a., Rifaximin Treatment in Hepatic Encephalopathy; N Engl J Med 2010; 362:1071-1081). Rifaximin is a semi-synthetic derivative of rifampin and acts by binding to the beta-subunit of bacterial DNA-dependent RNA polymerase, and thereby blocking transcription. As a result, bacterial protein synthesis and growth is inhibited.

Rifaximin has been shown to be active against E. coli both in vitro and in clinical studies. It therefore is understood that, for a combination treatment with rifaximin to be effective, the genetically engineered bacteria must further comprise a rifaximin resistance.

Resistance to rifaximin is caused primarily by mutations in the rpoB gene. This changes the binding site on DNA dependent RNA polymerase and decreases rifaximin binding affinity, thereby reducing efficacy. In one embodiment, the rifaximin resistance is a mutation in the rpoB gene. Non-limiting examples of such mutations are described in e.g., Rodriguez-Verdugo, Evolution of Escherichia coli rifampicin resistance in an antibiotic-free environment during thermal stress. BMC Evol Biol. 2013 Feb. 22; 13:50. Of note, mutations in the same three codons of the rpoB consensus sequence occur repeatedly in unrelated rifaximin-resistant clinical isolates of several different bacterial species (as reviewed in Goldstein, Resistance to rifampicin: a review; The Journal of Antibiotics (2014), 1-6, the contents of which is herein incorporated by reference in its entirety. In some embodiments, the genetically engineered bacteria comprise a known rifaximin resistance mutation, e.g., in the rpoB gene. In other embodiments, a screen can be employed, exposing the genetically engineered bacteria to increasing amounts of rifaximin, to identify a useful mutation which confers rifaximin resistance.

In some embodiments, the pharmaceutical composition is administered with food. In alternate embodiments, the pharmaceutical composition is administered before or after eating food. The pharmaceutical composition may be administered in combination with one or more dietary modifications, e.g., low-protein diet and amino acid supplementation. The dosage of the pharmaceutical composition and the frequency of administration may be selected based on the severity of the symptoms and the progression of the disorder. The appropriate therapeutically effective dose and/or frequency of administration can be selected by a treating clinician.

Treatment In Vivo

The genetically engineered bacteria of the invention may be evaluated in vivo, e.g., in an animal model. Any suitable animal model of a disease or condition associated with hyperammonemia may be used (see, e.g., Deignan et al., 2008; Nicaise et al., 2008), for example, a mouse model of acute liver failure and hyperammonemia. This acute liver failure and hyperammonemia may be induced by treatment with thiol acetamide (TAA) (Basile et al., 1990; Nicaise et al., 2008). Alternatively, liver damage may be modeled using physical bile duct ligation (Rivera-Mancía et al., 2012). Hyperammonemia may also be induced by oral supplementation with ammonium acetate and/or magnesium chloride (Azorin et al., 1989; Rivera-Mancía et al., 2012).

Additionally, CCl4 is often used to induce hepatic fibrosis and cirrhosis in animals (Nhung et al., Establishment of a standardized mouse model of hepatic fibrosis for biomedical research; Biomedical Research and Therapy 2014, 1(2):43-49).

The genetically engineered bacteria of the invention may be administered to the animal, e.g., by oral gavage, and treatment efficacy determined, e.g., by measuring ammonia in blood samples and/or arginine, citrulline, or other byproducts in fecal samples.

Full citations for the references cited throughout the specification include:
1. Alifano et al. Histidine biosynthetic pathway and genes: structure, regulation, and evolution. Microbiol Rev. 1996 March; 60(1):44-69. PMID: 8852895.
2. Altenhoefer et al. The probiotic Escherichia coli strain Nissle 1917 interferes with invasion of human intestinal epithelial cells by different enteroinvasive bacterial pathogens. FEMS Immunol Med Microbiol. 2004 Apr. 9; 40(3):223-229. PMID: 15039098.
3. Andersen et al. Uracil uptake in Escherichia coli K-12: isolation of uraA mutants and cloning of the gene. J Bacteriol. 1995 April; 177(8):2008-2013. PMID: 7721693.
4. Arthur et al. Intestinal inflammation targets cancer-inducing activity of the microbiota. Science. 2012 Oct. 5; 338(6103):120-123. PMID: 22903521.
5. Aoyagi et al. Gastrointestinal urease in man. Activity of mucosal urease. Gut. 1966 December; 7(6):631-635. PMID: 5957514.
6. Arai et al. Expression of the nir and nor genes for denitrification of Pseudomonas aeruginosa requires a novel CRP/FNR-related transcriptional regulator, DNR, in addition to ANR. FEBS Lett. 1995 Aug. 28; 371(1):73-76. PMID: 7664887.
7. Aschner et al. Manganese uptake and distribution in the central nervous system (CNS). Neurotoxicology. 1999 April-June; 20(2-3):173-180. PMID: 10385881.
8. Azorin et al. A simple animal model of hyperammonemia. Hepatology. 1989 September; 10(3):311-314. PMID: 2759549.
9. Bansky et al. Reversal of hepatic coma by benzodiazepene antagonists (Rol5-1788). Lancet. 1985; 1:1324-1325.
10. Basile et al. Brain concentrations of benzodiazepines are elevated in an animal model of hepatic encephalopathy. Proc Natl Acad Sci USA. 1990 July; 87(14):5263-5267. PMID: 1973539.
11. Bearden S W, Perry R D. The Yfe system of Yersinia pestis transports iron and manganese and is required for full virulence of plague. Mol Microbiol. 1999 April; 32(2):403-414. PMID: 10231495.
12. Berk D P, Chalmers T. Deafness complicating antibiotic therapy of hepatic encephalopathy. Ann Intern Med. 1970 September; 73(3):393-396. PMID: 5455989.
13. Blanc et al. Lactitol or lactulose in the treatment of chronic hepatic encephalopathy: results of a meta-analysis. Hepatology. 1992 February; 15(2):222-228. PMID: 1531204.
14. Caldara et al. The arginine regulon of Escherichia coli: whole-system transcriptome analysis discovers new genes and provides an integrated view of arginine regulation. Microbiology. 2006 November; 152(Pt 11):3343-3354. PMID: 17074904.
15. Caldara et al. Arginine biosynthesis in Escherichia coli: experimental perturbation and mathematical modeling. J Biol Chem. 2008 Mar. 7; 283(10):6347-6358. PMID: 18165237.
16. Caldovic et al. N-acetylglutamate synthase: structure, function and defects. Mol Genet Metab. 2010; 100 Suppl 1:S13-S19. Review. PMID: 20303810.
17. Callura et al. Tracking, tuning, and terminating microbial physiology using synthetic riboregulators. Proc Natl Acad Sci USA. 2010 Sep. 7; 107(36):15898-15903. PMID: 20713708.
18. Cash et al. Current concepts in the assessment and treatment of hepatic encephalopathy. QJM. 2010 January; 103(1):9-16. PMID: 19903725.
19. Castiglione et al. The transcription factor DNR from Pseudomonas aeruginosa specifically requires nitric oxide and haem for the activation of a target promoter in Escherichia coli. Microbiology. 2009 September; 155(Pt 9):2838-2844. PMID: 19477902.
20. Cellier et al. Resistance to intracellular infections: comparative genomic analysis of Nramp. Trends Genet. 1996 June; 12(6):201-204. PMID: 8928221.
21. Charlier et al. Arginine regulon of Escherichia coli K-12. A study of repressor-operator interactions and of in vitro binding affinities versus in vivo repression. J Mol Biol. 1992 Jul. 20; 226(2):367-386. PMID: 1640456.
22. Chiang et al. Dysregulation of C/EBPalpha by mutant Huntingtin causes the urea cycle deficiency in Huntington's disease. Hum Mol Genet. 2007 Mar. 1; 16(5):483-498. PMID: 17213233.
23. Collinson et al. Channel crossing: how are proteins shipped across the bacterial plasma membrane? Philos Trans R Soc Lond B Biol Sci. 2015; 370:20150025. PMID: 26370937.
24. Cordoba J, Mínguez B. Hepatic Encephalopathy. Semin Liver Dis. 2008; 28(1):70-80. PMID: 18293278.
25. Costa et al. Secretion systems in Gram-negative bacteria: structural and mechanistic insights. Nat Rev Microbiol. 2015; 13(6):343-359. PMID: 25978706.
26. Crabeel et al. Characterization of the Saccharomyces cerevisiae ARG7 gene encoding ornithine acetyltransferase, an enzyme also endowed with acetylglutamate synthase activity. Eur J Biochem. 1997 Dec. 1; 250(2):232-241. PMID: 9428669.
27. Cuevas-Ramos et al. Escherichia coli induces DNA damage in vivo and triggers genomic instability in mammalian cells. Proc Natl Acad Sci USA. 2010 Jun. 22; 107(25):11537-42. PMID: 20534522.
28. Cunin et al. Molecular basis for modulated regulation of gene expression in the arginine regulon of Escherichia coli K-12. Nucleic Acids Res. 1983 Aug. 11; 11(15):5007-5019. PMID: 6348703.
29. Cunin et al. Biosynthesis and metabolism of arginine in bacteria. Microbiol Rev. 1986 September; 50(3):314-52. Review. Erratum in: Microbiol Rev. 1987 March; 51(1):178. PMID: 3534538.
30. Danino et al. Programmable probiotics for detection of cancer in urine. Sci Transl Med. 2015 May 27; 7(289):289ra84. PMID: 26019220.
31. Deignan et al. Contrasting features of urea cycle disorders in human patients. Mol Genet Metab. 2008 January; 93(1):7-14. PMID: 17933574.
32. Deutscher. The mechanisms of carbon catabolite repression in bacteria. Curr Opin Microbiol. 2008 April; 11(2):87-93. PMID: 18359269.
33. Diaz et al. Ammonia control and neurocognitive outcome among urea cycle disorder patients treated with glycerol phenylbutyrate. Hepatology. 2013 June; 57(6):2171-9. PMID: 22961727.
34. Dinleyici et al. Saccharomyces boulardii CNCM I-745 in different clinical conditions. Expert Opin Biol Ther. 2014 November; 14(11):1593-609. PMID: 24995675.
35. Doolittle. A new allele of the sparse fur gene in the mouse. J Hered. 1974 May-June; 65(3):194-5. PMID: 4603259.
36. Eckhardt et al. Isolation and characterization of mutants with a feedback resistant N-acetylglutamate synthase in Escherichia coli K 12. Mol Gen Genet. 1975 Jun. 19; 138(3):225-32. PMID: 1102931.
37. Eiglmeier et al. Molecular genetic analysis of FNR-dependent promoters. Mol Microbiol. 1989 July; 3(7):869-78. PMID: 2677602.
38. Fraga et al. (2008). Real-Time PCR. Current Protocols Essential Laboratory Techniques (10.3.1-10.3.33). John Wiley & Sons, Inc.
39. Galimand et al. Positive FNR-like control of anaerobic arginine degradation and nitrate respiration in Pseudomonas aeruginosa. J Bacteriol. 1991 March; 173(5):1598-606. PMID: 1900277.
40. Gamper et al. Anaerobic regulation of transcription initiation in the arcDABC operon of Pseudomonas aeruginosa. J Bacteriol. 1991 August; 173(15):4742-50. PMID: 1906871.
41. Gardner et al. Construction of a genetic toggle switch in Escherichia coli. Nature. 2000; 403:339-42. PMID: 10659857.
42. Gorke B et al. Carbon catabolite repression in bacteria: many ways to make the most out of nutrients. Nat Rev Microbiol. 2008 August; 6(8):613-24. PMID: 18628769.
43. Häberle et al. Suggested guidelines for the diagnosis and management of urea cycle disorders. Orphanet J Rare Dis. 2012 May 29; 7:32. Review. PMID: 22642880.
44. Häberle J. Clinical and biochemical aspects of primary and secondary hyperammonemic disorders. Arch Biochem Biophys. 2013 Aug. 15; 536(2):101-8. Review. PMID: 23628343.
45. Hasegawa et al. Activation of a consensus FNR-dependent promoter by DNR of Pseudomonas aeruginosa in response to nitrite. FEMS Microbiol Lett. 1998 Sep. 15; 166(2):213-7. PMID: 9770276.
46. Hodges et al. The spfash mouse: a missense mutation in the ornithine transcarbamylase gene also causes aberrant mRNA splicing. Proc Natl Acad Sci USA. 1989 June; 86(11):4142-6. PMID: 2471197.
47. Hoeren et al. Sequence and expression of the gene encoding the respiratory nitrous-oxide reductase from Paracoccus denitrificans. Eur J Biochem. 1993 Nov. 15; 218(1):49-57. PMID: 8243476.
48. Hoffmann et al. Defects in amino acid catabolism and the urea cycle. Handb Clin Neurol. 2013; 113:1755-73. Review. PMID: 23622399.
49. Hosseini et al. Proprionate as a health-promoting microbial metabolite in the human gut. Nutr Rev. 2011 May; 69(5):245-58. PMID: 21521227.
50. Isabella et al. Deep sequencing-based analysis of the anaerobic stimulon in Neisseria gonorrhoeae. BMC Genomics. 2011 Jan. 20; 12:51. PMID: 21251255.
51. Konieczna et al. Bacterial urease and its role in long-lasting human diseases. Curr Protein Pept Sci. 2012 December; 13(8):789-806. Review. PMID: 23305365.
52. Lazier et al. Hyperammonemic encephalopathy in an adenocarcinoma patient managed with carglumic acid. Curr Oncol. 2014 October; 21(5):e736-9. PMID: 25302046.
53. Leonard (2006). Disorders of the urea cycle and related enzymes. Inborn Metabolic Diseases, 4^{th} ed (pp. 263-272). Springer Medizin Verlag Heidelberg.
54. Lim et al. Nucleotide sequence of the argR gene of Escherichia coli K-12 and isolation of its product, the arginine repressor. Proc Natl Acad Sci USA. 1987 October; 84(19):6697-701. PMID: 3116542.
55. Makarova et al. Conservation of the binding site for the arginine repressor in all bacterial lineages. Genome Biol. 2001; 2(4). PMID: 11305941.
56. Maas et al. Studies on the mechanism of repression of arginine biosynthesis in Escherichia coli. Dominance of repressibility in diploids. J Mol Biol. 1964 March; 8:365-70. PMID: 14168690.
57. Maas. The arginine repressor of Escherichia coli. Microbiol Rev. 1994 December; 58(4):631-40. PMID: 7854250.
58. Meng et al. Nucleotide sequence of the Escherichia coli cad operon: a system for neutralization of low extracellular pH. J Bacteriol. 1992 April; 174(8):2659-69. PMID: 1556085.
59. Moore et al. Regulation of FNR dimerization by subunit charge repulsion. J Biol Chem. 2006 Nov. 3; 281(44):33268-75. PMID: 16959764.
60. Mountain et al. Cloning of a Bacillus subtilis restriction fragment complementing auxotrophic mutants of eight Escherichia coli genes of arginine biosynthesis. Mol Gen Genet. 1984; 197(1):82-9. PMID: 6096675.
61. Nagamani et al. Optimizing therapy for argininosuccinic aciduria. Mol Genet Metab. 2012 September; 107(1-2):10-4. Review. PMID: 22841516.
62. Nicaise et al. Control of acute, chronic, and constitutive hyperammonemia by wild-type and genetically engineered Lactobacillus plantarum in rodents. Hepatology. 2008 October; 48(4):1184-92. PMID: 18697211.
63. Nicoloff et al. Two arginine repressors regulate arginine biosynthesis in Lactobacillus plantarum. J Bacteriol. 2004 September; 186(18):6059-69. PMID: 15342575.
64. Nougayrede et al. Escherichia coli induces DNA double-strand breaks in eukaryotic cells. Science. 2006 Aug. 11; 313(5788):848-51. PMID: 16902142.
65. Olier et al. Genotoxicity of Escherichia coli Nissle 1917 strain cannot be dissociated from its probiotic activity. Gut Microbes. 2012 November-December; 3(6):501-9. PMID: 22895085.
66. Pham et al. Multiple myeloma-induced hyperammonemic encephalopathy: an entity associated with high in-patient mortality. Leuk Res. 2013 October; 37(10):1229-32. Review. PMID: 23932549.
67. Rajagopal et al. Use of inducible feedback-resistant N-acetylglutamate synthetase (argA) genes for enhanced arginine biosynthesis by genetically engineered Escherichia coli K-12 strains. Appl Environ Microbiol. 1998 May; 64(5):1805-11. PMID: 9572954.
68. Ray et al. The effects of mutation of the anr gene on the aerobic respiratory chain of Pseudomonas aeruginosa. FEMS Microbiol Lett. 1997 Nov. 15; 156(2):227-32. PMID: 9513270.
69. Reister et al. Complete genome sequence of the Gram-negative probiotic Escherichia coli strain Nissle 1917. J Biotechnol. 2014 Oct. 10; 187:106-7. PMID: 25093936.
70. Rembacken et al. Non-pathogenic Escherichia coli versus mesalazine for the treatment of ulcerative colitis: a randomised trial. Lancet. 1999 Aug. 21; 354(9179):635-9. PMID: 10466665.
71. Remington's Pharmaceutical Sciences, 22^{nd} ed. Mack Publishing Co. 72. Salmon et al. Global gene expression profiling in Escherichia coli K12. The effects of oxygen availability and FNR. J Biol Chem. 2003 Aug. 8; 278(32):29837-55. PMID: 12754220.
73. Sat et al. The Escherichia coli mazEF suicide module mediates thymineless death. J Bacteriol. 2003 March; 185(6):1803-7. PMID: 12618443.
74. Sawers. Identification and molecular characterization of a transcriptional regulator from Pseudomonas aeruginosa PAO1 exhibiting structural and functional similarity to the FNR protein of Escherichia coli. Mol Microbiol. 1991 June; 5(6):1469-81. PMID: 1787797.
75. Schneider et al. Arginine catabolism and the arginine succinyltransferase pathway in Escherichia coli. J Bacteriol. 1998 August; 180(16): 4278-86. PMID: 9696779.
76. Schultz. Clinical use of E. coli Nissle 1917 in inflammatory bowel disease. Inflamm Bowel Dis. 2008 July; 14(7):1012-8. Review. PMID: 18240278.
77. Sonnenborn et al. The non-pathogenic Escherichia coli strain Nissle 1917—features of a versatile probiotic. Microbial Ecology in Health and Disease. 2009; 21:122-58.
78. Suiter et al. Fitness consequences of a regulatory polymorphism in a seasonal environment. Proc Natl Acad Sci USA. 2003 Oct. 28; 100(22):12782-6. PMID: 14555766.
79. Summerskill. On the origin and transfer of ammonia in the human gastrointestinal tract. Medicine (Baltimore). 1966 November; 45(6):491-6. PMID: 5925900.
80. Szwajkajzer et al. Quantitative analysis of DNA binding by the Escherichia coli arginine repressor. J Mol Biol. 2001 Oct. 5; 312(5):949-62. PMID: 11580241.
81. Tian et al. Binding of the arginine repressor of Escherichia coli K12 to its operator sites. J Mol Biol. 1992 Jul. 20; 226(2):387-97. PMID: 1640457.
82. Tian et al. Explanation for different types of regulation of arginine biosynthesis in Escherichia coli B and Escherichia coli K12 caused by a difference between their arginine repressors. J Mol Biol. 1994 Jan. 7; 235(1):221-30. PMID: 8289243.
83. Torres-Vega et al. Delivery of glutamine synthetase gene by baculovirus vectors: a proof of concept for the treatment of acute hyperammonemia. Gene Ther. 2014 Oct. 23; 22(1):58-64. PMID: 25338921.
84. Trunk et al. Anaerobic adaptation in Pseudomonas aeruginosa: definition of the Anr and Dnr regulons. Environ Microbiol. 2010 June; 12(6):1719-33. PMID: 20553552.
85. Tuchman et al. Enhanced production of arginine and urea by genetically engineered Escherichia coli K-12 strains. Appl Environ Microbiol. 1997 January; 63(1):33-8. PMID: 8979336.
86. Ukena et al. Probiotic Escherichia coli Nissle 1917 inhibits leaky gut by enhancing mucosal integrity. PLoS One. 2007 Dec. 12; 2(12):e1308. PMID: 18074031.
87. Unden et al. Alternative respiratory pathways of Escherichia coli: energetics and transcriptional regulation in response to electron acceptors. Biochim Biophys Acta. 1997 Jul. 4; 1320(3):217-34. Review. PMID: 9230919.
88. Vander Wauven et al. Pseudomonas aeruginosa mutants affected in anaerobic growth on arginine: evidence for a four-gene cluster encoding the arginine deiminase pathway. J Bacteriol. 1984 December; 160(3):928-34. PMID: 6438064.
89. Walker. Severe hyperammonaemia in adults not explained by liver disease. Ann Clin Biochem. 2012 May; 49(Pt 3):214-28. Review. PMID: 22349554.
90. Winteler et al. The homologous regulators ANR of Pseudomonas aeruginosa and FNR of Escherichia coli have overlapping but distinct specificities for anaerobically inducible promoters. Microbiology. 1996 March; 142 (Pt 3):685-93. PMID: 8868444.
91. Wu et al. Direct regulation of the natural competence regulator gene tfoX by cyclic AMP (cAMP) and cAMP receptor protein in Vibrios. Sci Rep. 2015 Oct. 7; 5:14921. PMID: 26442598.
92. Zimmermann et al. Anaerobic growth and cyanide synthesis of Pseudomonas aeruginosa depend on anr, a regulatory gene homologous with fnr of Escherichia coli. Mol Microbiol. 1991 June; 5(6):1483-90. PMID: 1787798.
93. Wright O, Delmans M, Stan G B, Ellis T. GeneGuard: A modular plasmid system designed for biosafety. ACS Synth Biol. 2015 Mar. 20; 4(3):307-16. PMID: 24847673.
94. Liu Y, White R H, Whitman W B. Methanococci use the diaminopimelate aminotransferase (DapL) pathway for lysine biosynthesis. J Bacteriol. 2010 July; 192(13):3304-10. PMID: 20418392.
95. Dogovski et al. (2012) Enzymology of Bacterial Lysine Biosynthesis, Biochemistry, Prof. Deniz Ekinci (Ed.), ISBN: 978-953-51-0076-8, InTech, Available from:
96. http://www.intechopen.com/books/biochemistry/enzymology-of-bacterial-lysine-biosynthesis.
97. Feng et al. Role of phosphorylated metabolic intermediates in the regulation of glutamine synthetase synthesis in Escherichia coli. J Bacteriol. 1992 October; 174(19):6061-70. PMID: 1356964.
98. Lodeiro et al. Robustness in Escherichia coli glutamate and glutamine synthesis studied by a kinetic model. J Biol Phys. 2008 April; 34(1-2):91-106. PMID: 19669495.
99. Reboul et al. Structural and dynamic requirements for optimal activity of the essential bacterial enzyme dihydrodipicolinate synthase. PLoS Comput Biol. 2012; 8(6):e1002537. PMID: 22685390.
100. Saint-Girons et al. Structure and autoregulation of the metJ regulatory gene in Escherichia coli. J Biol Chem. 1984 Nov. 25; 259(22):14282-5. PMID: 6094549.
101. Shoeman et al. Regulation of methionine synthesis in Escherichia coli: Effect of metJ gene product and S-adenosylmethionine on the expression of the metF gene. Proc Natl Acad Sci USA. 1985 June; 82(11):3601-5. PMID: 16593564.
102. van Heeswijk et al. Nitrogen assimilation in Escherichia coli: putting molecular data into a systems perspective. Microbiol Mol Biol Rev. 2013 December; 77(4):628-95. PMID: 24296575.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

Construction of plasmids encoding ammonia consuming circuits, including circuits comprising ΔArgR, ArgAfbr, and/or ΔThyA are inter alia described in the Examples of co-owned WO2017139697 and US20160333326, the contents of which is herein incorporated by reference in its entirety. A Functional Assay Demonstrating that the Recombinant Bacterial Cells disclosed herein consume ammonia and produce arginine is inter alia described in the Examples of co-owned WO2017139697 and US20160333326, the contents of which is herein incorporated by reference in its entirety. The in vitro activity of various strains (i.e., including ΔArgR and ArgA^{fbr} plus or minus ΔThyA) is described in the Examples of co-owned WO2017139697 and US20160333326. In vivo activity assays which may be used to determine in vivo efficacy for any of the strains described herein, e, are described in Examples of WO2017139697 and US20160333326 the contents of which is herein incorporated by reference in its entirety. Integration of constructs into the genome, e.g., using lambda red recombination is also described in WO2017139697 and US20160333326.

### Example 1. Pseudomonas taetrolens Racemase Expressed on a Low-Copy Plasmid is Functional

To determine whether Pseudomonas taetrolens racemase can convert L-arginine to D-arginine, in vitro D- and L-arginine production was compared in wild type Nissle, and arginine producing strains, which either did or did not include a tet-inducible Pseudomonas taetrolens arginine racemase (arR) circuit expressed from a low copy plasmid. The following strains were used in the study: (1) E. coli Nissle (SYN94), (2) ammonia consuming (arginine producing) strain (SYN-UCD824) comprising a deletion in argR, and tetracycline-inducible argA^{fbr} integrated into the chromosome at the malEK site, and (3) ammonia consuming (arginine producing) strain derived from SYN-UCD824 which further comprises a tet-inducible arginine racemase from Pseudomonas taetrolens (arR) on a low copy plasmid (SYN-UCD3230).

Briefly, cells were grown overnight in LB. Cells were then diluted 1/100 in LB, allowed to grow for 2 h at 37° C., shaking at 250 rpm, followed by 2 h induction (100 ng/mL ATC). Cells were spun down, washed and resuspended in M9, 0.5% glucose, at 1e9 cells/mL (using the cellometer). For the assay, cells were incubated at 37° C., 250 rpm for 3 h. Samples were taken at T0, h and 2 h and DL-arg quantified by LC-MS/MS.

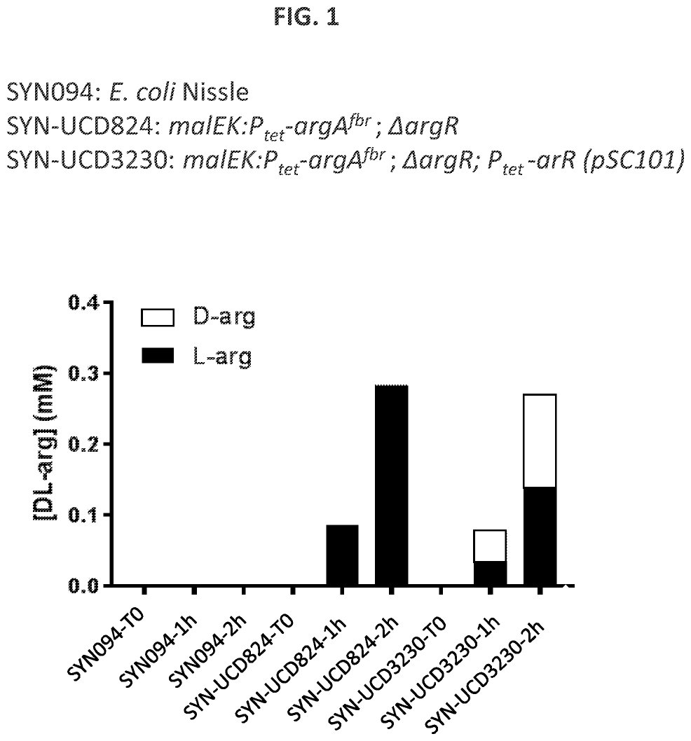

Results are shown in FIG. 1, showing that Pseudomonas taetrolens racemase expressed on a low-copy plasmid is functional.

### Example 2. Removal of the Signal Peptide for Export to Periplasm does not Impair Racemase Activity

Pseudomonas taetrolens arginine racemase is naturally localized to the periplasm, but deletion of a signal sequence allows expression of Pseudomonas taetrolens racemase in the cytosol, as was shown in C. glutamicum (Stabler et al., Corynebacterium glutamicum as a Host for Synthesis and Export of D-Amino Acids; J. Bacteriol. April 2011 vol. 193 no. 7 1702-1709).

In order to allow conversion of the L-arginine produced by the genetically engineered ammonia consuming strains to D-arginine in the cytoplasm of the bacterium, the signal peptide was removed, and the in vitro activity of the racemase without the signal peptide was tested. The following strains were used in this study: (1) E. coli Nissle (SYN94), (2) an ammonia consuming (arginine producing) strain (SYN-UCD824) comprising a deletion in argR, and tetracycline inducible argA^{fbr} integrated into the chromosome at the malEK site, and (3) an ammonia consuming (arginine producing) strain derived from SYN-UCD824, which further comprises, on a low copy plasmid, a tet-inducible arginine racemase from Pseudomonas taetrolens, which has a truncation in the first 23 AA of the polypeptide (arRΔ1-69), removing the signal sequence for export from the cell into the periplasm (SYN-UCD3331).

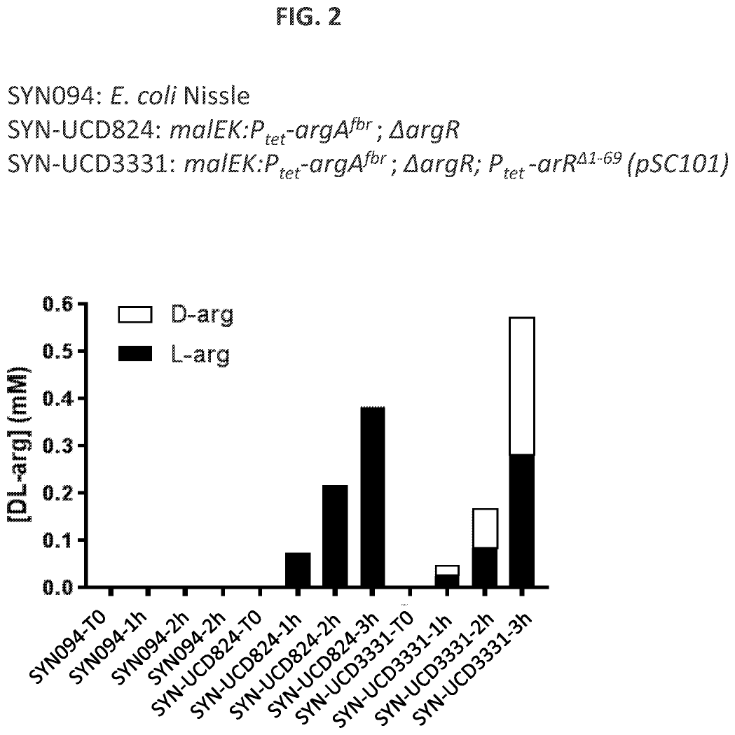

Cells were grown and L- and D-arginine production measured as described in the previous example. Results are shown in FIG. 2 and demonstrate that that truncating the first 23 AA (the signal peptide for export to periplasm) does not impair racemase activity

### Example 3. Measurement of Racemase Activity Using an Integrated, FNR Inducible Construct

The activity of a racemase integrated in tandem with ArgAfbr under the control of the FNR promoter was assessed. The following strains were used in this study: (1) SYN-UCD305 comprises ΔargR, feedback resistant argA under the control of an FNR promoter, and a thyA auxotrophy (ΔthyA); (2) SYN-UCD3649 comprises arRΔ1-69 under the control of an FNR promoter, and ΔargR and ΔthyA; (3) SYN-UCD3650 comprises argAfbr and arRΔ1-69 arranged in tandem, both under the control of an FNR promoter, ΔargR, and ΔthyA.

Briefly, overnight cultures were diluted 1/100 in LB, and allowed to grow for 2 hours at 37° C., 250 rpm. Next cells were induced for 2 hours without shaking. Cells were spun down, washed and resuspended in M9, 0.5% glucose, and 10 mM thymidine, at 1e9 cells/mL (using the cellometer). Cells were incubated at 37 C, 250 rpm for 3 h, and samples were taken at T0, 1 h, 2 h or 3 h. D- and L-arginine were quantified by LC-MS/MS.

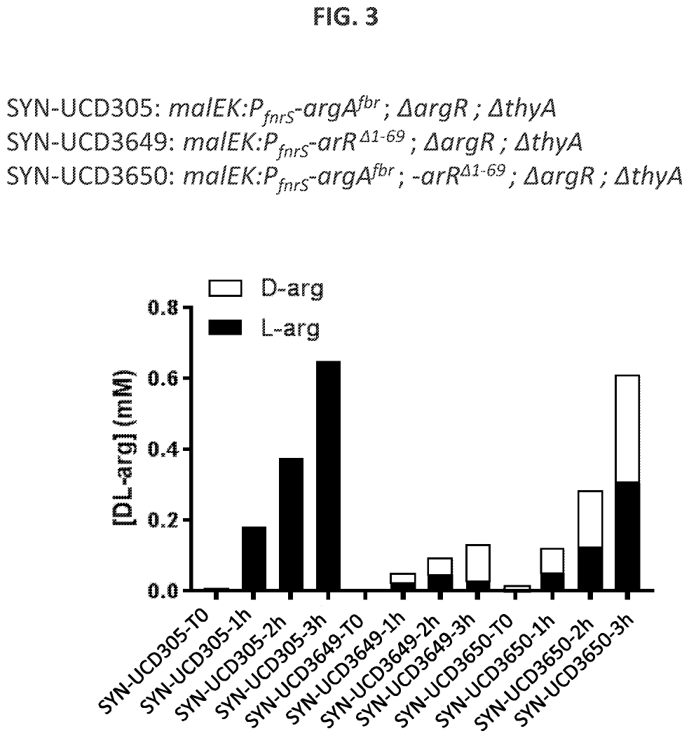

Results are shown in FIG. 3 and show that the racemase is active upon anaerobic induction, in particular in the L-arginine producing strain (which produces the substrate for the reaction). In the arginine producing strain containing the racemase, approximately half of the arginine is in the L-form and half is in the D-form, consistent with a racemase which is catalyzes the reaction in both directions.

### Example 4. Evaluation of Racemase Integrated Strains with Anaerobic Induction in C57BL/6J Mice

To determine whether the racemase is active in vivo and whether the racemase is useful for the detection of in vivo activity of the circuitry engineered into ammonia consuming strain SYN-UCD305 (P_{fnrS}-argA^{fbr} integrated at MalE/K, ΔargR, and ΔthyA), the activity of strains SYN-UCD3645 (malEK:P_{fnrS}-arRΔ1-69) and SYN-UCD3650 (malEK:P_{fnrs}-argA^{fbr}-arRΔ1-69; ΔargR; ΔthyA; essentially SYN-UCD305 circuitry plus racemase) was assessed in mice gavaged with the strains followed by urine collection and D-arginine measurements.

To prepare the cells for the study, overnight cultures were each used to inoculate a 125 mL ultra yield flask containing 12.5 mL of growth media, 40 g/L glycerol, 20 mM MOPS with 10 mM thymidine. Cultures were grown for 4 hours at 37 C, 350 rpm and then moved to static condition for 2 h. Cells were then spun down at 4000×g, washed with PBS once, re-centrifuged and concentrated 10× in PBS containing 15% glycerol before being placed at −80° C. for storage. On the day of gavage, cells were thawed on ice and sodium bicarbonate added to a final 100 mM concentration. On Day 0, animals were weighed and randomized into groups of 9 and fasted overnight. On day 1, animals were moved to metabolic caging for 1 hour and t=0 urine sample was collected. Animals were dosed with Wild-type C57B6 mice were dosed orally with 1e10 CFUs (100 ul) and placed in metabolic cages for collection of urine over 8 hours. Urine was collected from metabolic caging and by free catch at each time point. Samples were collected into pre-weighed 2 mL tubes and urine volume recorded/Samples will be kept on ice immediately after collection until frozen at −80° C. pending LC-MS/MS analysis.

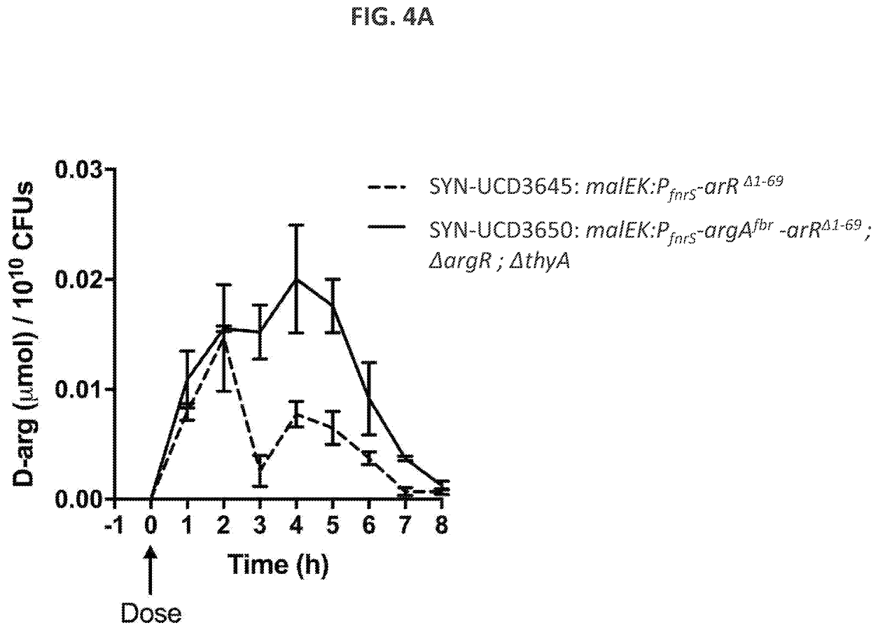

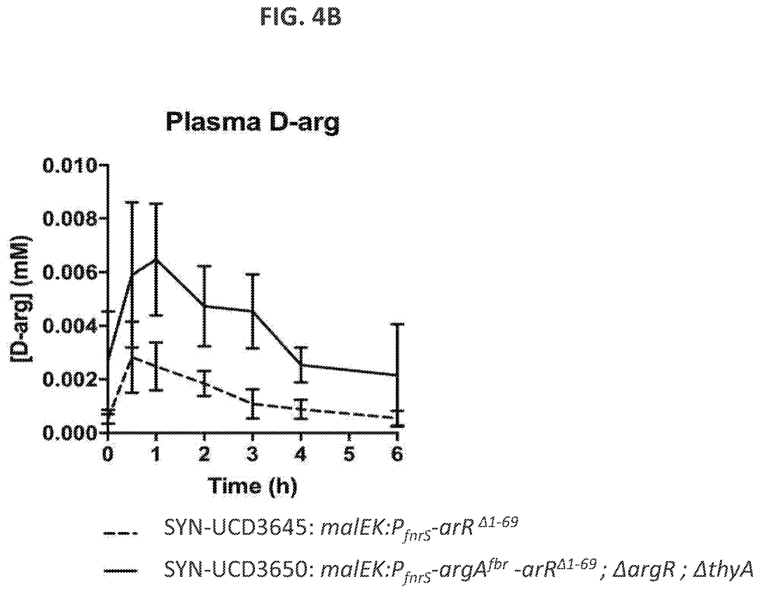

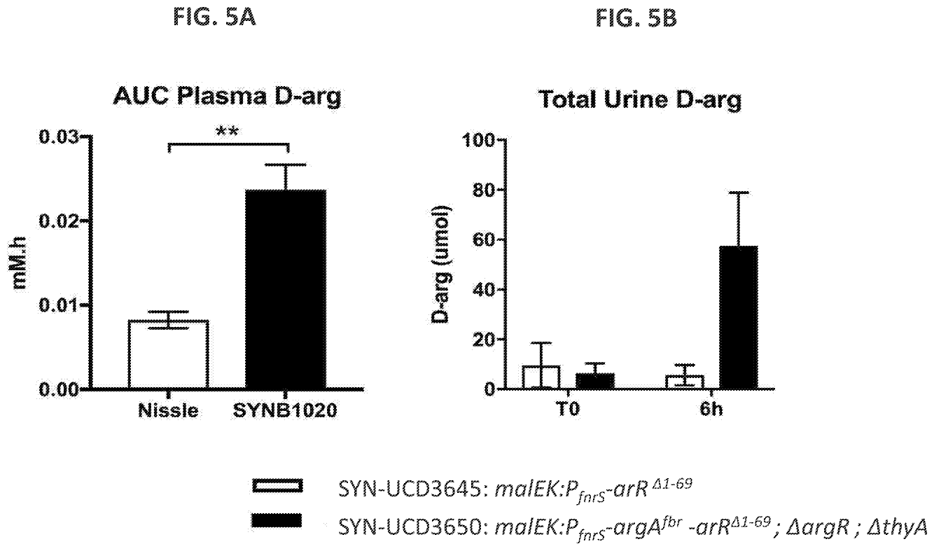

As shown in FIG. 4A, C57B6 mice dosed with the racemase containing SYN-UCD305 strain, excrete D-arginine in urine for over 5 hours following a single oral dose and have elevated plasma D-arg levels over a period of 6 hours (FIG. 4B). The observation of D-arginine in the urine indicates that the racemase is working in vivo. Additionally, the fact that more D-arg is excreted upon gavage with the strain engineered to produce L-arg than upon gavage with Nissle expressing the racemase alone indicates (1) that the L-arg biosynthesis pathway engineered in SYN-UCD305 (and SYN-UCD3650) is functional in vivo and (2) that D-arginine measurements as a result of the racemase activity are useful as a biomarker for strain activity.

In this study, results show that the strain containing SYN-UCD305 circuitry plus racemase (SYN-UCD3650) is active in vivo for at least 5 hours following a single oral dose, based on excretion of the biomarker D-arg in a modified SYN-UCD305 strain.

In this study, results show that the strain containing SYN-UCD305 circuitry plus racemase (SYN-UCD3650) is active in vivo for at least 5 hours following a single oral dose, based on excretion of the biomarker D-Arg in a modified SYN-UCD305 strain.

### Example 5. Efficacy of Genetically Engineered Bacteria in a Mouse Model of Hyperammonemia and Acute Liver Failure

Wild-type C57BL6/J mice are treated with thiol acetamide (TAA), which causes acute liver failure and hyperammonemia (Nicaise et al., 2008). The TAA mouse model is an industry-accepted in vivo model for HE. Mice are treated with unmodified control Nissle bacteria or Nissle bacteria engineered to produce high levels of arginine or citrulline as described above.

On day 1, 50 mL of the bacterial cultures of SYN-UCD3650 and control strains are grown overnight and pelleted. The pellets are resuspended in 5 mL of PBS at a final concentration of approximately 10¹¹ CFU/mL. Blood ammonia levels in mice are measured by mandibular bleed, and ammonia levels are determined by the PocketChem Ammonia Analyzer (Arkray). Mice are gavaged with 100 μL of bacteria (approximately 10¹⁰ CFU). Drinking water for the mice is changed to contain 0.1 mg/mL anhydrotetracycline (ATC) and 5% sucrose for palatability.

On day 2, the bacterial gavage solution is prepared as described above, and mice are gavaged with 100 μL of bacteria. The mice continue to receive drinking water containing 0.1 mg/mL ATC and 5% sucrose.

On day 3, the bacterial gavage solution is prepared as described above, and mice are gavaged with 100 μL of bacteria. The mice continue to receive drinking water containing 0.1 mg/mL ATC and 5% sucrose. Mice receive an intraperitoneal (IP) injection of 100 μL of TAA (250 mg/kg body weight in 0.5% NaCl).

On day 4, the bacterial gavage solution is prepared as described above, and mice are gavaged with 100 μL of bacteria. The mice continue to receive drinking water containing 0.1 mg/mL ATC and 5% sucrose. Mice receive another IP injection of 100 μL of TAA (250 mg/kg body weight in 0.5% NaCl). Blood ammonia levels in the mice are measured by mandibular bleed, and ammonia levels are determined by the PocketChem Ammonia Analyzer (Arkray).

On day 5, blood ammonia levels in mice are measured by mandibular bleed, and ammonia levels are determined by the PocketChem Ammonia Analyzer (Arkray). Fecal pellets are collected from mice to determine arginine content by liquid chromatography-mass spectrometry (LC-MS). Ammonia levels in mice treated with genetically engineered Nissle and unmodified control Nissle are compared.

Urine is collected and levels of D- and L-Arginine are determined.

### Example 6. Efficacy of Genetically Engineered Bacteria in a Mouse Model of Hyperammonemia and UCD

Ornithine transcarbamylase is urea cycle enzyme, and mice comprising an spf-ash mutation exhibit partial ornithine transcarbamylase deficiency, which serves as a model for human UCD. Mice are treated with unmodified control Nissle bacteria or Nissle bacteria engineered to produce high levels of arginine or citrulline or bacteria comprising ammonia consumption and D-arginine production circuitry as described herein.

60 spf-ash mice are treated with the genetically engineered bacteria of the invention or H2O control at 100 ul PO QD: H2O control, normal chow (n=15); H2O control, high protein chow (n=15); SYN-UCD3650, high protein chow (n=15); engineered bacteria, high protein chow (n=15). On Day 1, mice are weighed and sorted into groups to minimize variance in mouse weight per cage. Mice are gavaged and water with 20 mg/L ATC is added to the cages. On day 2, mice are gavaged in the morning and afternoon. On day 3, mice are gavaged in the morning and weighed, and blood is drawn 4 h post-dosing to obtain baseline ammonia levels. Mice are gavaged in the afternoon and chow changed to 70% protein chow. On day 4, mice are gavaged in the morning and afternoon. On day 5, mice are gavaged in the morning and weighed, and blood is drawn 4 h post-dosing to obtain ammonia levels. On days 6 and 7, mice are gavaged in the morning. On day 8, mice are gavaged in the morning and weighed, and blood is drawn 4 h post-dosing to obtain ammonia levels. On day 9, mice are gavaged in the morning and afternoon. On day 10, mice are gavaged in the morning and weighed, and blood is drawn 4 h post-dosing to obtain ammonia levels. On day 12, mice are gavaged in the morning and afternoon. On day 13, mice are gavaged in the morning and weighed, and blood is drawn 4 h post-dosing to obtain ammonia levels. Blood ammonia levels, body weight, and survival rates are analyzed. Urine is collected daily at various time points up to 8 hours post gavage and levels of D- and L-Arginine are determined.

### Example 7: SYN-UCD305: A Genetically Modified E. Coli Nissle Consumes Ammonia in a Mouse Model of UCD

The intestine is a major source of systemic ammonia (NH3), thus capturing part of gut NH3 may mitigate disease symptoms in conditions resulting from hyperammonemia, such as Urea Cycle Disorders (UCD). As a therapeutic strategy, E. coli Nissle (EcN), a well-characterized probiotic, was engineered to convert NH3 to L-arginine (L-arg) in the intestine by deleting a negative regulator of L-arg biosynthesis and expressing a feedback-resistant L-arg biosynthetic enzyme, SYN-UCD305. The essential gene thyA was also deleted to render SYN-UCD305 auxotrophic in the intestine and the environment. A modified version of SYN-UCD305 was created to convert L-arg to D-arg as a biomarker to follow activity of the strain in mice in vivo. SYN-UCD305 was evaluated in healthy mice and Otcspf-ash mice (defective in the urea cycle) for tolerability, excretion profile and NH₃ lowering activity.

SYN-UCD305 was evaluated for NH₃ consumption and L-arg production in vitro. A modified version of SYN-UCD305 capable of converting L-arg to D-arg was created through insertion of an arginine racemase derived from Pseudomonas taetrolens. This strain was evaluated to follow the duration of in vivo activity through urinary excretion of D-arg in mice. Wild-type C57BL6 mice were dosed orally with 1010 CFUs of this strain and placed in metabolic cages for collection of urine and feces over 8 hours. Urine and feces were analyzed for biomarkers including D-arg and L-arg. SYN-UCD305 was dosed twice daily in CD-1 mice for 28 days as part of a GLP toxicology study. The microbial kinetics of SYN-UCD305 excretion in feces were followed using qPCR with primers specific to EcN. The efficacy of a range of doses of SYN-UCD305 from 10⁹-10¹⁰ CFU were tested in in Otcspf-ash mice made hyperammonemic by placing them on a high protein diet. NH₃ levels and survival were monitored.

Results: SYN-UCD305 produced L-arg at a rate of 0.33 μmol/109 cells/hour and consumed NH3 at a rate of 1 μmol/109 cells/hour in an in vitro system. C57BL6 mice dosed with the racemate containing SYN-UCD305 strain, excreted D-arg in urine for over 5 hours following a single oral dose. SYN-UCD305 was well tolerated in CD-1 mice dosed twice daily for 28 days. SYN-UCD305 DNA was detectable in feces using a sensitive and specific qPCR method and reached peak levels in feces within 1 week of dosing. Following cessation of dosing, SYN-UCD305 declined rapidly and was undetectable in feces by 7 days post-dosing. SYN-UCD305 was able to reduce systemic hyperammonemia caused by a high-protein diet in Otcspf-ash mice at dose levels of 5×10⁹ and 1×10¹⁰ CFU/day. All mice treated with 5×10⁹ or 1×10¹⁰ CFUs were alive at 24 hours post treatment compared to only 40% of control-treated hyperammonemic mice and 50% of mice treated with 1×10⁹ CFUs.

Conclusions: SYN-UCD305 is designed to consume NH3 through production of L-arg and lowers systemic ammonia in Otcspf-ash mice, a genetic model of UCD. SYN-UCD305 is active in vivo for at least 5 hours following a single oral dose, based on excretion of the biomarker D-arg in a modified SYN-UCD305 strain. Based on NH3 lowering potential and safety and tolerability in mice, SYN-UCD305 should be further evaluated for therapeutic potential in patients UCD.

### Example 8. Quantification of L-Arg and D-Arg in Biological Samples by LC-MS/NIS

Sample Preparation

D-Arginine and L-Arginine standards (1000, 500, 250, 100, 20, 4, and 0.8 μg/mL) were prepared in LC-MS grade water. For QC samples, 750, 75, and 7.5 ug/mL were prepared. Samples were thawed on ice, and spun down at 4° C. Supernatants (10 μL) were from the spun down samples or 10 μL of L-arginine standards to a V-bottom 96-well plate. 90 μL of L-Arginine-¹³C₆,¹⁵N₄ internal standard solution (5 μg/mL L-Arginine-¹³C₆, ¹⁵N₄ dissolved in 70% LC-MS grade acetonitrile/30% LC-MS grade water) was added to samples and standards. The plate was heat-sealed with a PierceASeal seal and mix using a 96-well plate thermomixer for 5 sec at 400 rpm, and the centrifuged from the previous step at 4,000 rpm for 5 min at 4° C. 80 μL of the supernatants were transferred from the plate of the previous step to a fresh 96-well round-bottom polypropylene plate. Samples were dried under N₂ gas at 30° C. for 20 min at a flow rate of 30 L/min. The plate was then centrifuged at 4,000 rpm for 5 min at 4° C. or until about 5-10 μL was left. 100 μL of 50% Ethanol/LC-MS grade water was added to the samples in the above plate, while pipetting to mix. The plate was heat-sealed with a ClearASeal seal and samples were mixed by using a 96-well plate thermomixer for 5 sec at 400 rpm.

### LC-MS/MS Method

L- and D-Arginine were measured by liquid chromatography coupled to tandem mass spectrometry (LC-MS/MS) using a Thermo TSQ Quantum Max triple quadrupole mass spectrometer. The summary of the LC-MS/MS method is as follows:

Column: Sigma Astec Chirobiotic T column, 5 μm (150×2 mm)

Isocratic Mobile Phase: 70% Methanol

Flow rate: 1 mL/min

Injection volume: 10 uL

Run time: 12 min

### Tandem Mass Spectrometry:

Ion Source: HESI-II

Polarity: Positive

SRM transitions:
L-Arginine: 175.2/70.3
D-Arginine: 175.2/70.3 (more retained)
L-Arginine-¹³C₆, ¹⁵N₄: 175.1/75.35

### Example 9. Pharmacokinetics of a Genetically Engineered E. coli Nissle Following Administration to Non-Human Primates with PK Time Points

The objective of this study was to evaluate the production of arginine and metabolites by engineered bacteria or a control strain following oral administration in non-human primates. Bacteria evaluated are identified in Table 2.

TABLE 2
Test and Control Bacteria Identification

| | Test Article | Test Article | Test Article |
|---|---|---|---|
| Identification | Bacteria | Bacteria | Sodium |
| | | | Bicarbonate |
| Batch ID | SYN-UCD3650 | SYN-UCD3645 | |
| Concentration | 1.11 × 10¹¹ | 1.07 × 10¹¹ | 0.36M |
| | CFU/mL | CFU/mL | |
| Physical Description | Liquid | Liquid | liquid |
| Storage Conditions | −80° C. | −80° C. | 4° C. |

Bacteria (SYN-UCD3650 and SYN-UCD3645) were stored at −80° C. until use. Just before use, bacteria were thawed rapidly in a water bath at 37° C. and stored on wet ice until time of dosing animals Day 1. Experimental design is shown in Table 3.

TABLE 3
Experimental Design

| | No. | | Dose Volume | Dose |
|---|---|---|---|---|
| Day | Animals | Treatment | (mL) | Regimen |
| 1 | 5 | Sodium Bicarbonate | 5 | PO |
| | | Bacteria (SYN-UCD3650) | 10 | |
| | 5 | Sodium Bicarbonate | 5 | |
| | | Bacteria (SYN-UCD3645) | 10 | |

Briefly, bacteria were administered to the appropriate animals by oral gavage on Day 1. Dose formulations were administered by oral gavage using a disposable catheter attached to a plastic syringe in the following order: bicarbonate (5 mL) and bacteria (10 mL). Following dosing, the gavage tube was rinsed with 5 mL of the animal drinking water, into the animal's stomach. Each animal was dosed with a clean gavage tube. The first day of dosing was designated as Day 1 Animals were fasted overnight, and food was returned following the final blood collection after each dosing session.

Blood was collected by venipuncture and collected into K₂ EDTA tubes. After collection, samples were transferred to the appropriate laboratory for processing or stored at −70° C. Blood was collected from an appropriate peripheral vein other that the one used for dosing and was collected according to the following table.

TABLE 4
PK Sample Collection Schedule

| | Sample Collection Time Points (Time Post dose) on Day 1 | | | | | | |
|---|---|---|---|---|---|---|---|
| Group No. | 0^{a} hr | 30 min | 1 hr | 2 hr | 3 hr | 4 hr | 6 hr |
| 1 | X | X | X | X | X | X | X |

Samples were placed on crushed wet ice until subsequent centrifugation per standard procedures. The resultant plasma was separated, transferred to clear polypropylene tubes, and frozen immediately over dry ice and transferred to a freezer set to maintain −80° C.

For urine collection, animals were be separated, cage cleaned, and a clean collection pan inserted approximately 16-18 hours prior to dose to assist in urine collection at room temperature. At baseline (prior to dose) and conclusion of 6 hours post dose, the total amount of urine was measured, recorded, and a 2.5 mL in two aliquot samples was collected and frozen on dry ice. Samples were stored at −80° C.

TABLE 5
Sequences

| Description | Sequence |
|---|---|
| pFNRS-arginine | ggtaccAGTTGTTCTTATTGGTGGTGTTGCTTTATGGTTGCATCGTAGTAAAT |
| racemase_d1-23 | GGTTGTAACAAAAGCAATTTTTCCGGCTGTCTGTATACAAAAACGCCGCA |
| (RBS and leader | AAGTTTGAGCGAAGTCAATAAACTCTCTACCCATTCAGGGCAATATCTCTC |
| region underlined; | TTggatccaaagtgaactctagaaataattagataactttaagaaggagatatacatatggcgccacccctgtcg |
| pFNRS in italics) | atgaccgacggcgtagctcaagtgaatacccaggacagcaatgcctgggtcgaaatcaataaagccgcgttcga |
| SEQ ID NO: 1 | gcacaacatacggactctgcaaaccgccctcgccggcaagtcgcagatctgcgccgtactcaaggcggatgcct |
| | atggccacggtatcggcttgttgatgccctcggtgatcgccatgggtgttccctgtgtcggtgtcgccagcaacga |
| | agaagcccgcgtcgtgcgcgagagcggtttcaagggtcaactgatacgcgtgcgcaccgctgccctgagcgaa |
| | ctggaagctgcactgccgtacaacatggaagagctggtgggcaacctggacttcgcggtcaaggccagcctgat |
| | tgccgaggatcacggtcgcccgctggtggtgcacctgggtctgaattccagcggcatgagccgtaacggagtgg |
| | acatgaccaccgctcagggccgtcgtgatgcggtagctatcaccaaggtgccaaacctggaagtgcgggcgat |
| | catgacccacttcgcggtcgaagatgctgccgacgtgcgtgccgggctcaaggccttcaatcagcaagcccaat |
| | ggctgatgaacgtggcccagcttgatcgcagcaagatcaccctgcacgcggccaactcgttcgccacactggag |
| | gtgcccgaatcgcatctggacatggtccgccccggcggcgcgctgttcggcgacaccgtaccgtcccacaccg |
| | agtacaagcgggtcatgcagttcaagtcccacgtggcgtcggtcaacagctaccccaagggcaacaccgtcggt |
| | tatggccgcacgtacaccctggggcgcgactcgcggctggccaacatcaccgtcggctactctgacggctaccg |
| | ccgcgcgtttaccaataaagggattgtgctgatcaacggccatcgcgtgccagtggtgggcaaagtctcgatgaa |
| | caccctgatggtggacgtcactgacgcgccggatgtgaaaagcggcgatgaagtggtgctgttcgggcaccag |
| | ggcaaggccgagattacccaggctgagatcgaagacatcaacggtgcactgcttgcggatctgtataccgtgtg |
| | gggcaattccaaccctaaaatcctgaaagatcagtaa |
| PFNRS with RBS | ggtaccAGTTGTTCTTATTGGTGGTGTTGCTTTATGGTTGCATCGTAGTA |
| and Leader region | AATGGTTGTAACAAAAGCAATTTTTCCGGCTGTCTGTATACAAAAAC |
| (RBS and leader | GCCGCAAAGTTTGAGCGAAGTCAATAAACTCTCTACCCATTCAGGGC |
| region underlined) | AATATCTCTCTTggatccaaagtgaactctagaaataattttgtttaactttaagaaggagatatacat |
| SEQ ID NO: 2 | |
| RBS and leader | ctctagaaataattttgtttaactttaagaaggagatatacat |
| region | |
| SEQ ID NO: 3 | |
| arginine | atggcgccacccctgtcgatgaccgacggcgtagctcaagtgaatacccaggacagcaatgcctgggtcgaaat |
| racemase_d1-23 | caataaagccgcgttcgagcacaacatacggactctgcaaaccgccctcgccggcaagtcgcagatctgcgcc |
| (first 69 bp of the | gtactcaaggcggatgcctatggccacggtatcggcttgttgatgccctcggtgatcgccatgggtgttccctgtgt |
| arR (arginine | cggtgtcgccagcaacgaagaagcccgcgtcgtgcgcgagagcggtttcaagggtcaactgatacgcgtgcgc |
| racemase), | accgctgccctgagcgaactggaagctgcactgccgtacaacatggaagagctggtgggcaacctggacttcg |
| corresponding to the | cggtcaaggccagcctgattgccgaggatcacggtcgcccgctggtggtgcacctgggtctgaattccagcggc |
| 23 AA signal | atgagccgtaacggagtggacatgaccaccgctcagggccgtcgtgatgcggtagctatcaccaaggtgccaa |
| peptide, have been | acctggaagtgcgggcgatcatgacccacttcgcggtcgaagatgctgccgacgtgcgtgccgggctcaaggc |
| deleted) | cttcaatcagcaagcccaatggctgatgaacgtggcccagcttgatcgcagcaagatcaccctgcacgcggcca |
| SEQ ID NO: 4 | actcgttcgccacactggaggtgcccgaatcgcatctggacatggtccgccccggcggcgcgctgttcggcgac |
| | accgtaccgtcccacaccgagtacaagcgggtcatgcagttcaagtcccacgtggcgtcggtcaacagctaccc |
| | caagggcaacaccgtcggttatggccgcacgtacaccctggggcgcgactcgcggctggccaacatcaccgtc |
| | ggctactctgacggctaccgccgcgcgtttaccaataaagggattgtgctgatcaacggccatcgcgtgccagtg |
| | gtgggcaaagtctcgatgaacaccctgatggtggacgtcactgacgcgccggatgtgaaaagcggcgatgaag |
| | tggtgctgttcgggcaccagggcaaggccgagattacccaggctgagatcgaagacatcaacggtgcactgctt |
| | gcggatctgtataccgtgtggggcaattccaaccctaaaatcctgaaagatcagtaa |
| arginine | MAPPLSMTDGVAQVNTQDSNAWVEINKAAFEHNIRTLQTALAGKSQIC |
| racemase_d1-23 | AVLKADAYGHGIGLLMPSVIAMGVPCVGVASNEEARVVRESGFKGQLI |
| (polypeptide) first | RVRTAALSELEAALPYNMEELVGNLDFAVKASLIAEDHGRPLVVHLGL |
| 69 bp of the arR | NSSGMSRNGVDMTTAQGRRDAVAITKVPNLEVRAIMTHFAVEDAADV |
| (arginine racemase), | RAGLKAFNQQAQWLMNVAQLDRSKITLHAANSFATLEVPESHLDMVR |
| corresponding to the | PGGALFGDTVPSHTEYKRVMQFKSHVASVNSYPKGNTVGYGRTYTLG |
| 23 AA signal | RDSRLANITVGYSDGYRRAFTNKGIVLINGHRVPVVGKVSMNTLMVDV |
| peptide, have been | TDAPDVKSGDEVVLFGHQGKAEITQAEIEDINGALLADLYTVWGNSNP |
| deleted) | KILKDQ* |
| SEQ ID NO: 5 | |
| pFNRS-fbr ArgA- | ggtaccAGTTGTTCTTATTGGTGGTGTTGCTTTATGGTTGCATCGTAGTAAAT |
| arginine racemase | GGTTGTAACAAAAGCAATTTITCCGGCTGTCTGTATACAAAAACGCCGCA |
| d1-23 | AAGTTTGAGCGAAGTCAATAAACTCTCTACCCATTCAGGGCAATATCTCTC |
| (RBS and Leader | TTggatccaaagtgaactctagaaataattttgtttaactttaagaaggagatatacatATGGTAAAGGA |
| region underlined, | ACGTAAAACCGAGTTGGTCGAGGGATTCCGCCATTCGGTTCCCTGTA |
| pFNRS in italics, | TCAATACCCACCGGGGAAAAACGTTTGTCATCATGCTCGGCGGTGA |
| racemase in bold) | AGCCATTGAGCATGAGAATTTCTCCAGTATCGTTAATGATATCGGGT |
| SEQ ID NO: 6 | TGTTGCACAGCCTCGGCATCCGTCTGGTGGTGGTCTATGGCGCACGT |
| | CCGCAGATCGACGCAAATCTGGCTGCGCATCACCACGAACCGCTGT |
| | ATCACAAGAATATACGTGTGACCGACGCCAAAACACTGGAACTGGT |
| | GAAGCAGGCTGCGGGAACATTGCAACTGGATATTACTGCTCGCCTGT |
| | CGATGAGTCTCAATAACACGCCGCTGCAGGGCGCGCATATCAACGT |
| | CGTCAGTGGCAATTTTATTATTGCCCAGCCGCTGGGCGTCGATGACG |
| | GCGTGGATTACTGCCATAGCGGGCGTATCCGGCGGATTGATGAAGA |
| | CGCGATCCATCGTCAACTGGACAGCGGTGCAATAGTGCTAATGGGG |
| | CCGGTCGCTGTTTCAGTCACTGGCGAGAGCTTTAACCTGACCTCGGA |
| | AGAGATTGCCACTCAACTGGCCATCAAACTGAAAGCTGAAAAGATG |
| | ATTGGTTTTTGCTCTTCCCAGGGCGTCACTAATGACGACGGTGATAT |
| | TGTCTCCGAACTTTTCCCTAACGAAGCGCAAGCGCGGGTAGAAGCCC |
| | AGGAAGAGAAAGGCGATTACAACTCCGGTACGGTGCGCTTTTTGCG |
| | TGGCGCAGTGAAAGCCTGCCGCAGCGGCGTGCGTCGCTGTCATTTAA |
| | TCAGTTATCAGGAAGATGGCGCGCTGTTGCAAGAGTTGTTCTCACGC |
| | GACGGTATCGGTACGCAGATTGTGATGGAAAGCGCCGAGCAGATTC |
| | GTCGCGCAACAATCAACGATATTGGCGGTATTCTGGAGTTGATTCGC |
| | CCACTGGAGCAGCAAGGTATTCTGGTACGCCGTTCTCGCGAGCAGCT |
| | GGAGATGGAAATCGACAAATTCACCATTATTCAGCGCGATAACACG |
| | ACTATTGCCTGCGCCGCGCTCTATCCGTTCCCGGAAGAGAAGATTGG |
| | GGAAATGGCCTGTGTGGCAGTTCACCCGGATTACCGCAGTTCATCAA |
| | GGGGTGAAGTTCTGCTGGAACGCATTGCCGCTCAGGCTAAGCAGAG |
| | CGGCTTAAGCAAATTGTTTGTGCTGACCACGCGCAGTATTCACTGGT |
| | TCCAGGAACGTGGATTTACCCCAGTGGATATTGATTTACTGCCCGAG |
| | AGCAAAAAGCAGTTGTACAACTACCAGCGTAAATCCAAAGTGTTGA |
| | TGGCGGATTTAGGGTAAtaagaaggagatatacatatggcgccacccctgtcgatgaccgacg |
| | gcgtagctcaagtgaatacccaggacagcaatgcctgggtcgaaatcaataaagccgcgttcgagcaca |
| | acatacggactctgcaaaccgccctcgccggcaagtcgcagatctgcgccgtactcaaggcggatgcctat |
| | ggccacggtatcggcttgttgatgccctcggtgatcgccatgggtgttccctgtgtcggtgtcgccagcaac |
| | gaagaagcccgcgtcgtgcgcgagagcggtttcaagggtcaactgatacgcgtgcgcaccgctgccctga |
| | gcgaactggaagctgcactgccgtacaacatggaagagctggtgggcaacctggacttcgcggtcaagg |
| | ccagcctgattgccgaggatcacggtcgcccgctggtggtgcacctgggtctgaattccagcggcatgagc |
| | cgtaacggagtggacatgaccaccgctcagggccgtcgtgatgcggtagctatcaccaaggtgccaaacc |
| | tggaagtgcgggcgatcatgacccacttcgcggtcgaagatgctgccgacgtgcgtgccgggctcaaggc |
| | cttcaatcagcaagcccaatggctgatgaacgtggcccagcttgatcgcagcaagatcaccctgcacgcg |
| | gccaactcgttcgccacactggaggtgcccgaatcgcatctggacatggtccgccccggcggcgcgctgtt |
| | cggcgacaccgtaccgtcccacaccgagtacaagcgggtcatgcagttcaagtcccacgtggcgtcggtc |
| | aacagctaccccaagggcaacaccgtcggttatggccgcacgtacaccctggggcgcgactcgcggctgg |
| | ccaacatcaccgtcggctactctgacggctaccgccgcgcgtttaccaataaagggattgtgctgatcaac |
| | ggccatcgcgtgccagtggtgggcaaagtctcgatgaacaccctgatggtggacgtcactgacgcgccgg |
| | atgtgaaaagcggcgatgaagtggtgctgttcgggcaccagggcaaggccgagattacccaggctgaga |
| | tcgaagacatcaacggtgcactgcttgcggatctgtataccgtgtggggcaattccaaccctaaaatcctga |
| | aagatcagtaa |
| fbr ArgA | ATGGTAAAGGAACGTAAAACCGAGTTGGTCGAGGGATTCCGCCATT |
| SEQ ID NO: 7 | CGGTTCCCTGTATCAATACCCACCGGGGAAAAACGTTTGTCATCATG |
| | CTCGGCGGTGAAGCCATTGAGCATGAGAATTTCTCCAGTATCGTTAA |
| | TGATATCGGGTTGTTGCACAGCCTCGGCATCCGTCTGGTGGTGGTCT |
| | ATGGCGCACGTCCGCAGATCGACGCAAATCTGGCTGCGCATCACCA |
| | CGAACCGCTGTATCACAAGAATATACGTGTGACCGACGCCAAAACA |
| | CTGGAACTGGTGAAGCAGGCTGCGGGAACATTGCAACTGGATATTA |
| | CTGCTCGCCTGTCGATGAGTCTCAATAACACGCCGCTGCAGGGCGCG |
| | CATATCAACGTCGTCAGTGGCAATTTTATTATTGCCCAGCCGCTGGG |
| | CGTCGATGACGGCGTGGATTACTGCCATAGCGGGCGTATCCGGCGG |
| | ATTGATGAAGACGCGATCCATCGTCAACTGGACAGCGGTGCAATAG |
| | TGCTAATGGGGCCGGTCGCTGTTTCAGTCACTGGCGAGAGCTTTAAC |
| | CTGACCTCGGAAGAGATTGCCACTCAACTGGCCATCAAACTGAAAG |
| | CTGAAAAGATGATTGGTTTTTGCTCTTCCCAGGGCGTCACTAATGAC |
| | GACGGTGATATTGTCTCCGAACTTTTCCCTAACGAAGCGCAAGCGCG |
| | GGTAGAAGCCCAGGAAGAGAAAGGCGATTACAACTCCGGTACGGTG |
| | CGCTTTTTGCGTGGCGCAGTGAAAGCCTGCCGCAGCGGCGTGCGTCG |
| | CTGTCATTTAATCAGTTATCAGGAAGATGGCGCGCTGTTGCAAGAGT |
| | TGTTCTCACGCGACGGTATCGGTACGCAGATTGTGATGGAAAGCGCC |
| | GAGCAGATTCGTCGCGCAACAATCAACGATATTGGCGGTATTCTGG |
| | AGTTGATTCGCCCACTGGAGCAGCAAGGTATTCTGGTACGCCGTTCT |
| | CGCGAGCAGCTGGAGATGGAAATCGACAAATTCACCATTATTCAGC |
| | GCGATAACACGACTATTGCCTGCGCCGCGCTCTATCCGTTCCCGGAA |
| | GAGAAGATTGGGGAAATGGCCTGTGTGGCAGTTCACCCGGATTACC |
| | GCAGTTCATCAAGGGGTGAAGTTCTGCTGGAACGCATTGCCGCTCAG |
| | GCTAAGCAGAGCGGCTTAAGCAAATTGTTTGTGCTGACCACGCGCA |
| | GTATTCACTGGTTCCAGGAACGTGGATTTACCCCAGTGGATATTGAT |
| | TTACTGCCCGAGAGCAAAAAGCAGTTGTACAACTACCAGCGTAAAT |
| | CCAAAGTGTTGATGGCGGATTTAGGGTAA |
| fbr ArgA | MVKERKTELVEGFRHSVPCINTHRGKTFVIMLGGEAIEHENFSSIVNDIG |
| SEQ ID NO: 8 | LLHSLGIRLVVVYGARPQIDANLAAHHHEPLYHKNIRVTDAKTLELVK |
| | QAAGTLQLDITARLSMSLNNTPLQGAHINVVSGNFIIAQPLGVDDGVDY |
| | CHSGRIRRIDEDAIHRQLDSGAIVLMGPVAVSVTGESFNLTSEEIATQLAI |
| | KLKAEKMIGFCSSQGVTNDDGDIVSELFPNEAQARVEAQEEKGDYNSG |
| | TVRFLRGAVKACRSGVRRCHLISYQEDGALLQELFSRDGIGTQIVMESA |
| | EQIRRATINDIGGILELIRPLEQQGILVRRSREQLEMEIDKFTIIQRDNTTIA |
| | CAALYPFPEEKIGEMACVAVHPDYRSSSRGEVLLERIAAQAKQSGLSKL |
| | FVLTTRSIHWFQERGFTPVDIDLLPESKKQLYNYQRKSKVLMADLG* |
| Pseudomonas | atgcccttctcccgtaccctgctcgccctttcccttggcatggcattgctgcaaaacccggcctttgctgcgccacc |
| taetrolens arR for | cctgtcgatgaccgacggcgtagctcaagtgaatacccaggacagcaatgcctgggtcgaaatcaataaagccg |
| arginine racemase | cgttcgagcacaacatacggactctgcaaaccgccctcgccggcaagtcgcagatctgcgccgtactcaaggcg |
| (with signal peptide) | gatgcctatggccacggtatcggcttgttgatgccctcggtgatcgccatgggtgttccctgtgtcggtgtcgccag |
| SEQ ID NO: 9 | caacgaagaagcccgcgtcgtgcgcgagagcggtttcaagggtcaactgatacgcgtgcgcaccgctgccctg |
| | agcgaactggaagctgcactgccgtacaacatggaagagctggtgggcaacctggacttcgcggtcaaggcca |
| | gcctgattgccgaggatcacggtcgcccgctggtggtgcacctgggtctgaattccagcggcatgagccgtaac |
| | ggagtggacatgaccaccgctcagggccgtcgtgatgcggtagctatcaccaaggtgccaaacctggaagtgc |
| | gggcgatcatgacccacttcgcggtcgaagatgctgccgacgtgcgtgccgggctcaaggccttcaatcagcaa |
| | gcccaatggctgatgaacgtggcccagcttgatcgcagcaagatcaccctgcacgcggccaactcgttcgccac |
| | actggaggtgcccgaatcgcatctggacatggtccgccccggcggcgcgctgttcggcgacaccgtaccgtcc |
| | cacaccgagtacaagcgggtcatgcagttcaagtcccacgtggcgtcggtcaacagctaccccaagggcaaca |
| | ccgtcggttatggccgcacgtacaccctggggcgcgactcgcggctggccaacatcaccgtcggctactctgac |
| | ggctaccgccgcgcgtttaccaataaagggattgtgctgatcaacggccatcgcgtgccagtggtgggcaaagt |
| | ctcgatgaacaccctgatggtggacgtcactgacgcgccggatgtgaaaagcggcgatgaagtggtgctgttcg |
| | ggcaccagggcaaggccgagattacccaggctgagatcgaagacatcaacggtgcactgcttgcggatctgtat |
| | accgtgtggggcaattccaaccctaaaatcctgaaagatcagtaa |
| Pseudomonas | tgcccttctcccgtaccctgctcgccctttcccttggcatggcattgctgcaaaacccggcctttgct |
| taetrolens arR for | |
| arginine racemase | |
| signal peptide | |
| SEQ ID NO: 10 | |
| Pseudomonas | gcgccacccctgtcgatgaccgacggcgtagctcaagtgaatacccaggacagcaatgcctgggtcgaaatca |
| taetrolens arR for | ataaagccgcgttcgagcacaacatacggactctgcaaaccgccctcgccggcaagtcgcagatctgcgccgta |
| arginine racemase | ctcaaggcggatgcctatggccacggtatcggcttgttgatgccctcggtgatcgccatgggtgttccctgtgtcg |
| minus signal peptide | gtgtcgccagcaacgaagaagcccgcgtcgtgcgcgagagcggtttcaagggtcaactgatacgcgtgcgcac |
| SEQ ID NO: 11 | cgctgccctgagcgaactggaagctgcactgccgtacaacatggaagagctggtgggcaacctggacttcgcg |
| | gtcaaggccagcctgattgccgaggatcacggtcgcccgctggtggtgcacctgggtctgaattccagcggcat |
| | gagccgtaacggagtggacatgaccaccgctcagggccgtcgtgatgcggtagctatcaccaaggtgccaaac |
| | ctggaagtgcgggcgatcatgacccacttcgcggtcgaagatgctgccgacgtgcgtgccgggctcaaggcctt |
| | caatcagcaagcccaatggctgatgaacgtggcccagcttgatcgcagcaagatcaccctgcacgcggccaact |
| | cgttcgccacactggaggtgcccgaatcgcatctggacatggtccgccccggcggcgcgctgttcggcgacac |
| | cgtaccgtcccacaccgagtacaagcgggtcatgcagttcaagtcccacgtggcgtcggtcaacagctacccca |
| | agggcaacaccgtcggttatggccgcacgtacaccctggggcgcgactcgcggctggccaacatcaccgtcgg |
| | ctactctgacggctaccgccgcgcgtttaccaataaagggattgtgctgatcaacggccatcgcgtgccagtggt |
| | gggcaaagtctcgatgaacaccctgatggtggacgtcactgacgcgccggatgtgaaaagcggcgatgaagtg |
| | gtgctgttcgggcaccagggcaaggccgagattacccaggctgagatcgaagacatcaacggtgcactgcttgc |
| | ggatctgtataccgtgtggggcaattccaaccctaaaatcctgaaagatcagtaa |
| Pseudomonas | MPFSRTLLALSLGMALLQNPAFAAPPLSMTDGVAQVNTQDSNAWVEIN |
| taetrolens arR for | KAAFEHNIRTLQTALAGKSQICAVLKADAYGHGIGLLMPSVIAMGVPC |
| arginine racemase | VGVASNEEARVVRESGFKGQLIRVRTAALSELEAALPYNMEELVGNLD |
| (with signal peptide) | FAVKASLIAEDHGRPLVVHLGLNSSGMSRNGVDMTTAQGRRDAVAITK |
| SEQ ID NO: 12 | VPNLEVRAIMTHFAVEDAADVRAGLKAFNQQAQWLMNVAQLDRSKIT |
| | LHAANSFATLEVPESHLDMVRPGGALFGDTVPSHTEYKRVMQFKSHVA |
| | SVNSYPKGNTVGYGRTYTLGRDSRLANITVGYSDGYRRAFTNKGIVLIN |
| | GHRVPVVGKVSMNTLMVDVTDAPDVKSGDEVVLFGHQGKAEITQAEI |
| | EDINGALLADLYTVWGNSNPKILKDQ* |
| Signal Peptide | MPFSRTLLALSLGMALLQNPAFA |
| SEQ ID NO: 13 | |
| Pseudomonas | AAPPLSMTDGVAQVNTQDSNAWVEINKAAFEHNIRTLQTALAGKSQIC |
| taetrolens arR for | AVLKADAYGHGIGLLMPSVIAMGVPCVGVASNEEARVVRESGFKGQLI |
| arginine racemase | RVRTAALSELEAALPYNMEELVGNLDFAVKASLIAEDHGRPLVVHLGL |
| minus signal peptide | NSSGMSRNGVDMTTAQGRRDAVAITKVPNLEVRAIMTHFAVEDAADV |
| SEQ ID NO: 14 | RAGLKAFNQQAQWLMNVAQLDRSKITLHAANSFATLEVPESHLDMVR |
| | PGGALFGDTVPSHTEYKRVMQFKSHVASVNSYPKGNTVGYGRTYTLG |
| | RDSRLANITVGYSDGYRRAFTNKGIVLINGHRVPVVGKVSMNTLMVDV |
| | TDAPDVKSGDEVVLFGHQGKAEITQAEIEDINGALLADLYTVWGNSNP |
| | KILKDQ* |

1. An engineered bacterium capable of reducing excess ammonia or capable of converting ammonia and/or nitrogen into an alternate byproduct, wherein the bacterium comprises a racemase.

2. The bacterium of claim 1, wherein the racemase is an amino acid racemase.

3. The bacterium of claim 2, wherein the amino acid racemase is an arginine racemase.

4. The bacterium of claim 1, wherein the racemase is a d1-23 racemase or an ArR racemase.

5. (canceled)

6. The bacterium of claim 1, wherein the racemase is from Pseudomonas taetrolens.

7. The bacterium of claim 1, wherein the racemase is selected from the group consisting of EC 5.1.1.1 (alanine racemase), EC 5.1.1.2 (methionine racemase), EC 5.1.1.3 (glutamine racemase), EC 5.1.1.4 (proline racemase), EC 5.1.1.5 (lysine racemase), EC 5.1.1.6 (threonine racemase), EC 5.1.1.7 (diaminopimelate epimerase), EC 5.1.1.8 (4-hydroxyproline epimerase), EC 5.1.1.9 (arginine racemase), EC 5.1.1.10 (amino acid racemase), EC 5.1.1.11 (phenylalanine racemase), EC 5.1.1.12 (ornithine racemase), EC 5.1.1.13 (aspartate racemase), EC 5.1.1.14 (nocardicin-A epimerase), EC 5.1.1.15 (2-aminohexano-6-lactam racemase), EC 5.1.1.16 (protein-serine racemase), EC 5.1.1.17 (isopenicillin-N racemase), and EC 5.1.1.18 (serine racemase).

8. The bacterium of claim 1, wherein the racemase does not comprise a signal peptide, or wherein the racemase does comprise a signal peptide.

9. (canceled)

10. The bacterium of claim 4, wherein the racemase comprises a sequence that is at least 90% identical to SEQ ID NO:5, SEQ ID NO:12, or SEQ ID NO:14.

11. The bacterium of claim 4, wherein the racemase is encoded by a sequence that is at least 90% identical to SEQ ID NO:4, SEQ ID NO:9, or SEQ ID NO:11.

12. The bacterium of claim 1, comprising a modification to lack a functional ArgR.

13.-14. (canceled)

15. The bacterium of claim 1, wherein the bacterium is an auxotroph in a gene that is complemented when the bacterium is present in a mammalian gut.

16. (canceled)

17. The bacterium of claim 1, wherein the bacterium further comprises an arginine feedback resistant N-acetylglutamate synthetase (ArgA^{fbr}).

18. A pharmaceutical composition comprising the engineered bacterium of claim 1.

19. A method of treating a subject, the method comprising administering the pharmaceutical composition of claim 18 to the subject, thereby treating the subject.

20. A method of decreasing ammonia levels in a subject, the method comprising administering the pharmaceutical composition of claim 18 to the subject, thereby decreasing ammonia levels in the subject.

21. The method of claim 19, further comprising collecting a urine and/or feces sample from the subject and measuring the level of D-arginine and/or L-arginine in the sample.

22. A method of monitoring the treatment of a subject and who has previously been administered the engineered bacterium of claim 1, the method comprising measuring levels of D-arginine and/or L-arginine in the urine and/or feces of the subject, thereby monitoring the treatment of the subject.

23. Use of the engineered bacterium of claim 1 as an indicator of the ability of the bacterium to reduce excess ammonia or convert ammonia and/or nitrogen into an alternate byproduct, wherein the use comprises measuring levels of D-arginine and/or L-arginine in the urine and/or feces of a subject who has previously been administered the engineered bacterium.

24. The method of claim 21, wherein an increased level of D-arginine in the urine and/or feces of the subject as compared to a control indicates that the engineered bacterium is reducing excess ammonia and/or converting ammonia and/or nitrogen into an alternate byproduct.

25. The method of claim 24, wherein the control is a level of D-arginine in the subject prior to administration of the engineered bacterium, or wherein the control is a level of D-arginine from a population of subjects not treated with the engineered bacterium.

26. (canceled)

27. The method of claim 25, wherein the level of D-arginine in the urine and/or feces of the subject is increased at least 1.5 fold as compared to the control, at least 2 fold as compared to the control, or at least 6 fold as compared to the control.

28.-30. (canceled)
